(19) United States
(12) Patent Application Publication
(10) Pub. No.: US 2015/0073535 A1
(43) Pub. Date: Mar. 12, 2015

(54) Treatment of coronary artery lesions with a scaffold having vessel scaffold interactions that reduce or prevent angina

(71) Applicant: Abbott Cardiovascular Systems Inc., Santa Clara, CA (US)

(72) Inventors: Paul Consigny, San Jose, CA (US); Richard J. Rapoza, San Francisco, CA (US); Syed Faiyaz Ahmed Hossainy, Hayward, CA (US); Chad J. Abunassar, San Francisco, CA (US); Alexander J. Sheehy, Redwood City, CA (US); Laura E. Perkins, Mattaponi, VA (US); Santosh V. Prabhu, Santa Clara, CA (US); Wai-Fung Cheong, Los Altos, CA (US); Pooja A. Sadarangani, Santa Clara, CA (US); Stephen D. Pacetti, San Jose, CA (US)

(21) Appl. No.: 14/120,555

(22) Filed: Jun. 2, 2014

Related U.S. Application Data
(60) Provisional application No. 61/877,241, filed on Sep. 12, 2013.
(60) Provisional application No. 61/895,961, filed on Oct. 25, 2013.

(51) Int. Cl. A61L 31/14; A61L 31/16; A61F 2/06

(52) U.S. Cl. CPC A61L 31/148; A61F 2/06; A61L 31/16; A61F 2002/065; A61F 2002/068
USPC 623/1.38

(57) ABSTRACT

Methods of treating coronary artery disease (CAD) with bioresorbable stents resulting in reduced angina or non-ischemic chest pain are described. Methods of treatment and devices for treatment of angina and post-procedural chest pain that include anti-angina agents incorporated into the device are disclosed.

This application claims the benefit of U.S. Patent Application Ser. No. 61/877,241 filed Sep. 12, 2013 and U.S. Patent Application Ser. No. 61/895,961 filed Oct. 25, 2013, both of which are incorporated by reference herein.

### BACKGROUND OF THE INVENTION

1. Field of the Invention

This invention relates to bioresorbable polymer scaffolds and methods of treatment of coronary lesions with bioresorbable polymer scaffolds

2. Description of the State of the Art

This invention relates generally to methods of treatment with radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffold gets its name because it physically holds open and, if desired, expands the wall of a passageway in a patient. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty.

Stents are used not only for mechanical intervention but also as vehicles for providing biological therapy. Biological therapy uses medicated stents to locally administer a therapeutic substance. The therapeutic substance can also mitigate an adverse biological response to the presence of the stent. A medicated stent may be fabricated by coating the surface of either a metallic or polymeric scaffolding with a polymeric carrier that includes an active or bioactive agent or drug. Polymeric scaffolding may also serve as a carrier of an active agent or drug.

The stent must be able to satisfy a number of mechanical requirements. The stent must have sufficient radial strength so that it is capable of withstanding the structural loads, namely radial compressive forces imposed on the stent as it supports the walls of a vessel. Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading, which if exceeded, creates a yield stress condition resulting in the stent diameter not returning to its unloaded diameter, i.e., there is irrecoverable deformation of the stent. When the radial yield strength is exceeded the stent is expected to yield more severely and only a minimal force is required to cause major deformation.

Once expanded, the stent must adequately ₚrovide lumen support during a time required for treatment in spite of the various forces that may come to bear on it, including the cyclic loading induced by the beating heart. In addition, the stent must possess sufficient flexibility with a certain resistance to fracture.

Stents made from biostable or non-degradable materials, such as metals that do not corrode or have minimal corrosion during a patient's lifetime, have become the standard of care for percutaneous coronary intervention (PCI) as well as in peripheral applications, such as the superficial femoral artery (SFA). Such stents have been shown to be capable of preventing early and later recoil and restenosis.

In order to affect healing of a diseased blood vessel, the presence of the stent is necessary only for a limited period of time, as the artery undergoes physiological remodeling over time after deployment. The development of a bioabsorbable stent or scaffold could obviate the permanent metal implant in vessel, allow late expansive luminal and vessel remodeling, and leave only healed native vessel tissue after the full resorption of the scaffold. Stents fabricated from bioresorbable, biodegradable, bioabsorbable, and/or bioerodable materials such as bioabsorbable polymers can be designed to completely absorb only after or some time after the clinical need for them has ended. Consequently, a fully bioabsorbable stent can reduce or eliminate the risk of potential long-term complications and of late thrombosis, facilitate non-invasive diagnostic MRI /CT imaging, allow restoration of normal vasomotion, and provide the potential for plaque regression.

### SUMMARY OF THE INVENTION

Embodiment one of the present invention includes a method of treating coronary artery disease (CAD) in a patient comprising: identifying a patient or population of patients in need of treatment of CAD; optionally identifying factors, conditions, characteristics, or any combination thereof of the patient or population of patients in need of treatment for CAD which makes the patient susceptible to angina or non-ischemic thoracic chest pain; optionally recommending treatment or describing advantages of treatment or describing advantages relating to reduced rate of angina following implantation of a bioresorbable stent for treating the CAD for patients; and implanting the bioresorbable stent in the patient or population of patients for treating CAD, wherein the stent is implanted at a lesion or stenotic portion of a blood vessel in the patient or population of patients, wherein the implanted stent treats the CAD and the patient or population of patients experiences no angina or non-ischemic thoracic chest pain or a reduced degree of angina non-ischemic thoracic chest pain as compared to a metal platform stent.

Advantages can refer to any or any combination of conditions resulting from treatment with the bioresorbable stent or mechanisms described as causing or contributing to the conditions.

The method of embodiment one may have one or more, or any combination of the following aspects (1) to (7): (1) the recommending is as an alternative to the metal platform stent; (2) the factors, conditions, or characteristics are selected from the group consisting of type of coronary lesion, suffering from a CAD-related condition or non-CAD disease, race, ethnicity, gender, and any combination thereof; (3) the type of coronary lesions is selected from the group consisting of bifurcated lesion, long diffuse lesion, ostial lesion, and vulnerable plaque suspect lesion (less than 50% occlusion by angiography); (4) the CAD-related condition or non-CAD disease is selected from the group consisting of suffering diabetes, obesity, and prone to vasospasm; (5) the race or ethnicity comprises Indian sub-continent descent; (6) the implanted scaffold during the first 30 days after implantation has mechanical interactions with the vessel which result in reduced stress and strain on the vessel as compared to a metallic stent which results in the reduced chest pain; and (7) the mechanical interactions of aspect (6) that results in the reduced stress include axial conformability, circumferential conformability, lower radial stiffness, reduced compression, or any combination thereof.

Embodiment two of the present invention includes a method of treating coronary artery disease (CAD) in a patient or population of patients comprising: identifying factors, conditions, or characteristics, or any combination thereof of a patient or population of patients in need of treatment for CAD which makes the patient or population of patients susceptible to angina; and recommending treatment or describing advantages relating to reduced angina following implantation of a bioresorbable stent for treating the CAD for patients with such factors, conditions, or characteristics.

Embodiment three of the present invention includes a method of treating coronary artery disease (CAD) in a patient comprising: identifying a bioresorbable stent; and recommending treatment or describing advantages relating to reduced rate of angina of the bioresorbable stent for treating the CAD for patients.

The method of embodiments two and three may have one or more, or any combination of the following aspects (1) to (7): (1) providing a plurality of the recommended bioresorbable stent to a health care provider that implants the stents in a population of patients and a statistically significant number of the population of patients experiences lower frequency, severity, or diagnosis rate of angina than has been shown for a metal platform stent; (2) the recommended bioresorbable stent has been shown to provide a reduced rate of angina as compared to a metal platform stent in other patient populations; (3) the recommendation is made as an alternative to a metal platform stent; (4) the advantages comprise reduced angina from treatment with the bioresorbable stent as compared to a metal platform stent; (5) providing or sending the bioresorbable stent to a medical facility, medical professional, or distributer for distribution to a medical facility or medical professional for treatment of a patient or population of patients in need of treatment of the CAD that has or does not have one or more of the factors, conditions, or characteristics; (6) implanting the bioresorbable stents in a patient or population of patients in need of treatment of the CAD that has or does not have one or more of the factors, conditions, or characteristics, wherein the implanted stent treats the CAD and the patient or population of patients experiences no angina or non-ischemic chest pain or a reduced degree of angina or non-ischemic chest pain as compared to a metallic stent ; and (7) the factors, conditions, or characteristics are selected from the group consisting type of coronary lesion, suffering from a CAD-related condition or non-CAD disease, race, ethnicity, gender, and any combination thereof.

Embodiment four of the present invention includes a method of treating coronary artery disease (CAD) in a patient or a population of patients comprising: identifying a patient or population of patients in need of treatment of CAD; optionally, identifying factors, conditions, characteristics, or any combination thereof of the patient in need of treatment for CAD which makes the patient or the population of patients susceptible to angina or non-ischemic chest pain; and implanting the bioresorbable stent in the patient or the population of patients for treating CAD, wherein the stent is implanted in a stenotic portion of a blood vessel of the patient or the population of patients, wherein the implanted stent treats the CAD and exhibits reduced stress-strain interactions with the vessel as compared to a metal platform stent due to greater axial conformability, circumferential conformity, reduced medial compression, or any combination thereof as compared to the metal platform stent, wherein the reduced stress-strain interaction contribute to reducing angina or non-ischemic chest pain in the patient or the population of patients experienced post-implantation as compared to the metal platform stent.

Embodiment five of the present invention includes a method of treating coronary artery disease (CAD) in a patient or a population of patients comprising: identifying a patient or the population of patients in need of treatment of CAD; recommending treatment or describing advantages of treatment of CAD with a bioresorbable polymer stent based on reduced stress-strain interactions with the vessel as compared to a metal platform stent due to greater axial conformability, circumferential conformity, lower radial stiffness, reduced medial compression, or any combination thereof as compared to the metal platform stent, wherein the reduced stress-strain interaction contribute to reducing angina or non-ischemic chest pain in the patient or the population of patients experienced by the patient or the population of patients post-implantation as compared to a metal platform stent.

Embodiment six of the present invention includes a method of treating coronary artery disease (CAD) in a patient or population of patients comprising: identifying a patient or population of patients in need of treatment of CAD; and implanting the bioresorbable polymer stent in the patient or population of patients for treating the CAD, wherein the stent is implanted in a stenotic segment of a blood vessel in the patient or population of patients, wherein during an first period of at least 30 days when the mechanical properties of the stent are unaffected by degradation the stent exhibits reduced stress-strain interactions with the vessel as compared to a metal platform stent due to greater axial conformability, circumferential conformity, reduced medial compression, or any combination thereof, wherein during a second period after the first period, stress-strain interactions with the vessel are reduced due to degradation of the stent resulting in a decrease in radial strength of the stent and loss of mechanical integrity of the stent both of which increase the vessel freedom of movement, wherein the increase in freedom of movement of the vessel allows for pulsatility in the vessel and optionally positive remodeling of the vessel during the second period, and wherein angina is reduced in the patient or the population of patients as compared to a metal platform stent or prevented during the first and/or second period due to one or any combination of the reduced stress-strain interactions in the first period, the increased pulsatility during the second period, and the positive remodeling during the second period.

The method of embodiment six may have one or more, or any combination of the following aspects (1) to (6): (1) the increased pulsatility and the positive remodeling additively enhance blood flow rate which reduces angina in the patient or the population of patients as compared to a metal platform stent, wherein the increased pulsatility and the positive remodeling increase the blood flow rate as compared with a reference flow rate for a stented vessel and wherein the increased pulsatility and the positive remodeling additively enhance blood flow rate between 6 and 12 months post-implantation; (2) the increased pulsatility enhances blood flow rate which reduces angina in the patient or the population of patients as compared to the metal platform stent prior to or in the absence of the positive remodeling, and wherein the increased pulsatility and reduction in angina starts at about 6 months; (3) the reduced strain-strain interactions in the first period provides optimal stress-strain equilibration during the first period which reduces angina in the patient or the population of patients as compared to a metal platform stent while maintaining patency; (4) the reduced strain-strain interactions in the first period and the increased pulsatility promote, additively or synergistically, functional neo media/endothelium, resulting in benign positive remodeling, and wherein the reduced strain-strain interactions in the first period and the increased pulsatility promote, additively or synergistically, functional neo media/endothelium starts at about 6 months post implantation; (5) a time dependent load bearing property of the bioresorbable stent allows for the stent to conform to the vessel during positive remodeling which is benign without malapposition resulting in the reduction or prevention of angina in the patient or the population of patients; and (6) aspects (1) to (6) follow a superposition principle and additively or synergistically contribute to reduction of angina in the patient or the population of patients.

Embodiment seven of the present invention includes a method of treating coronary artery disease (CAD) in a patient or a population of patients comprising: identifying a patient or a population of patients in need of treatment of CAD; identifying a bioresorbable stent and communicating one or more properties of the bioresorbable stent when implanted in a patient comprising:
P1—reduced degradation independent stress-strain interactions with the vessel due to increased radial and axial compliance of the stent as compared to a metal platform stent;
P2—increased vasomotion due to freedom of movement of the scaffold due to degradation of the mechanical properties of the stent;
P3—benign positive remodeling of the vessel;
P4—reduced focal wall stress beneath the struts due to a larger stent/artery area ratio for the bioresorbable stent compared to the metal platform stent; or
any combination thereof;

recommending treatment or describing advantages of treatment of CAD with a bioresorbable polymer stent based on:
(a) P2+P3 additively enhancing flow rate between about 6-12 months post implantation, thereby reducing angina;
(b) P2 enhancing flow rate starting at about 6 months post-implantation;
(c) P1 providing optimal stress-strain equilibration during vessel scaffolding at t=0;
(d) P1 and P2 promoting, additively or synergistically, functional neo media/endothelium starting at about 6 months post-implantation, resulting in benign positive remodeling;
(e) Degradation dependent load bearing property of the stent allowing for the scaffold to conform to the vessel during benign positive remodeling without malapposition of struts with the vessel wall;
(f) any combination of (a) to (e) following superposition principle and additively or synergistically contribute to reduction of angina at about 1 yr and optionally reduce target lesion revascularization (TLR) and major adverse cardiac event (MACE) at greater than 12 months post-implantation; or
(g) P1 contributing to acute safety.

The method of embodiment seven may have one or more, or any combination of the following aspects (1) to (6): (1) providing a plurality of the recommended bioresorbable stent to a health care provider that implants the stents in a population of patients and a statistically significant number of the population of patients experiences lower rate of angina than has been shown for a metal platform stent in other patient populations; (2) the recommended bioresorbable stent has been shown to provide a reduced rate of angina as compared to the metal platform stent in other patient populations; (3) the recommendation is made as an alternative to the metal platform stent; (4) the advantages comprise reduced angina from treatment with the bioresorbable polymer stent as compared to the metal platform stent; (5) providing or sending the bioresorbable stent to a medical facility, medical professional, or distributer for distribution to a medical facility or medical professional for treatment of a patient or population of patients in need of treatment of the CAD that has or does not have one or more of the factors, conditions, or characteristics that are indicators of angina; and (6) implanting the bioresorbable polymer stents in a patient or population of patients in need of treatment of the CAD that has or does not have one or more of the factors, conditions, or characteristics that are indicators of angina, wherein the implanted stent treats the CAD and the patient or population of patients experiences no angina or non-ischemic chest pain or a reduced degree of angina or non-ischemic chest pain as compared to the metal platform stent.

Embodiment eight of the present invention includes a method of treating coronary artery disease (CAD) in a patient comprising: implanting the bioresorbable stent in the patient or population of patients for treating CAD, wherein the stent is implanted at a lesion or stenotic portion of a blood vessel in the patient or population of patients, and wherein the implanted stent has been shown to result in reduced angina or non-ischemic thoracic chest pain as compared to a metallic stent.

The method of embodiment eight may have one or more, or any combination of the following aspects (1)-(2): (1) the reduced angina has been shown by reduced angina site diagnosis rate in clinical or non-clinical patient populations and (2) the bioresorbable stent comprises a PLLA-based scaffold.

Embodiment nine of the present invention includes a method of treating coronary artery disease (CAD) in a patient comprising: implanting a bioresorbable polymer stent in the patient or population of patients for treating CAD, wherein the stent is implanted at a lesion or stenotic portion of a blood vessel in the patient or population of patients, and wherein the implanted stent has been shown to have a site diagnosed angina (SDA) in a patient population of:
less than 8%, less than 6%, or 4% to 8% at 37 days (post-intervention) PI, 30 days, or 30 to 40 days PI, or
less than 12%, less than 14%, or 10% to 14% at 193 days PI, 180 days PI, or 175 to 195 days PI, or
less than 16%, less than 18%. 14% to 20% at 393 days PI, 1 year PI, or 365 to 400 days PI.

The method of embodiment nine may have one or more, or any combination of the following aspects (1)-(2): (1) the reduced angina is reduced SDA in clinical or non-clinical patient populations and (2) the bioresorbable stent comprises a PLLA-based scaffold.

Embodiment ten includes a medical device, comprising: a bioabsorbable stent body; and a coating layer, the coating layer comprising of a bioabsorbable coating polymer and an anesthetic agent. Embodiment eleven includes a method of reducing or eliminating post-procedural chest pain in a patient having a scaffold implanted in a blood vessel for treating coronary artery disease comprising releasing an amount of a local anesthetic agent from the scaffold effective to provide tissue concentrations of the local anesthetic agent in a wall of the blood vessel to act on nerves associated with chest pain causing a reversible loss of sensation that reduces or eliminates chest pain. In embodiment eleven, the anesthetic agent may be released from a coating layer on the scaffold comprising a bioabsorbable polymer and the anesthetic agent.

Embodiments ten and eleven may have one or more, or any combination of the following aspects (1)-(8): (1) a number average molecular weight of the bioabsorbable coating polymer is less than 200 kDa as measured by gel permeation chromatography using polystyrene standards; (2) the coating layer has a thickness selected from the group consisting of less than 5 microns, less than 3 microns, or 2 to 4 microns; (3) the coating and dose of the anesthetic agent provides a release of the anesthetic agent effective to reduce or eliminate post-procedural chest pain in the patient during at least the first two weeks post-implantation; (4) a dose per unit stent body length of the anesthetic agent on the stent body is selected from the group consisting of less than 1 μg/mm, 1 to 3 μg/mm, 3 to 5 μg/mm, 5 to 7 μg/mm, 7 to 10 μg/mm, and greater than 10 μg/mm; (5) the anesthetic agent is selected from the group consisting of lidocaine, mepivacaine, bupivacaine, levobupivacaine, ropivacaine, etidocaine, prilocaine, articaine, and any combination thereof; (6) the bioabsorbable stent body comprises a material selected from the group consisting of a lactide-based polymer, a bioerodible metal, and a tyrosine-based polycarbonate polymer; (7) at least 85% of the anesthetic is released at 1 month after implantation; (8) wherein a dose of the anesthetic agent varies along a length of the stent body such that the dose increases from a proximal to a distal end of the stent; and (9) the coating layer further comprises an antiproliferative agent.

Embodiment twelve includes a medical device, comprising: a bioabsorbable stent body; and a coating layer, the coating layer comprising of a bioabsorbable coating polymer and an anti-angina agent selected from the group consisting of nitrates, beta-blockers, calcium channel blockers, ranexa, nitric oxide donors, nitric oxide generators, and alpha-adrenergic blockade agents.

Embodiment thirteen includes a method of reducing or eliminating chronic angina in a patient having a scaffold implanted in a blood vessel for treating coronary artery disease during a period of two weeks to 12 months post scaffold implantation comprising releasing an amount of an anti-angina agent from the scaffold effective to provide a tissue concentrations of the agent at or adjacent to an implant site of the scaffold that reduces or eliminates ischemia associated with chest pain causing a reduction or elimination of chest pain and the anti-angina agent is selected from the group consisting of nitrates, beta-blockers, calcium channel blockers, ranexa, nitric oxide donors, nitric oxide generators, and alpha-adrenergic blockade agents.

In embodiment thirteen, the anti-angina agent may be released from a coating layer on a body of the scaffold and/or from the body of the scaffold comprising a bioabsorbable polymer and the anti-angina agent. In embodiment thirteen, a calcium channel blocker may reduce or eliminate ischemia associated with chest pain by disrupting the movement of calcium ions (Ca²⁺) through calcium channels from outside to the inside of smooth muscle cells locally at the implant site which reduces contraction of the blood vessel and causes an increase in diameter at the implant site. In embodiment thirteen, nitric oxide donors and generators may reduce or eliminate ischemia associated with chest pain by generation of nitric oxide upon breakdown/dissociation or catalyzing the generation of nitric oxide, respectively, the generated nitric oxide acting as a vasodilator, an inhibitor of smooth muscle cell migration/proliferation, and an inhibitor of platelet adhesion/aggregation at the implant site. In embodiment thirteen, alpha-adrenergic blockade agents may reduce or eliminate ischemia associated with chest pain by blocking alpha adrenergic-mediated arteriole vasoconstriction which blocks sympathetic tone and increases blood flow at the implant site.

Embodiments twelve and thirteen may have one or more, or any combination of the following aspects (1)-(7): (1) a number average molecular weight of the bioabsorbable coating polymer is less than 200 kDa; (2) the coating layer has a thickness selected from the group consisting of less than 5 microns, less than 3 microns, or 1 to 410 microns; (3) upon implantation of the medical device in a patient, the coating and dose of the anti-angina agent provides a release of the anti-angina agent effective to reduce or eliminate ischemia induced post-procedural chest pain in the patient for a duration of one to six months post-implantation, more preferably between oneand 12 months post-implantation; (4) a dose per unit stent body length of the anti-angina agent on the stent body is selected from the group consisting of less than 1 μg/mm, 1 to 3 μg/mm, 3 to 5 μg/mm, 5 to 7 μg/mm, 7 to 10 μg/mm, and 1 to 25 μg/mm; (5) less than 50% of the anti-angina agent is released at 6 months after implantation; (6) the coating layer further comprises an antiproliferative agent; (7) the calcium channel blockers are selectedfrom the group consisting of dihydropyridines, lacidipine, amlodipine, nicardipine, nefedipine, felodipine, and phenylalkylamines; and (8) wherein the anesthetic agent is released preferentially from a distal end of the scaffold.

Embodiments of aspect (7) include one or more, or any combination of the following aspects (7a) and (7b): (7a) the phenylalkylamines are selected from the group consisting of verapamil, gallopimil, and fendiline and (7b) the alpha adrenergic blockers are selected from the group consisting of non-selective alpha blockade agents and selective alpha blockade agents.

Embodiments of aspect (7b) include one or more, or any combination of the following aspects (7bi)-(7biii): (7bi) non-selective alpha adrenergic blockade agents are selected from the group consisting of phenoxybenzamine, phentolamine, trazodone, and tolazine; (7bii) the selective blockade agents are selective for alpha-1 blockage and are selected from the group consisting of prazosin and doxazosin; and (7biii) the selective blockade agents are selective for alpha-2 blockage and are selected from the group consisting of idazoxan, and yohimbine.

Embodiment fourteen of the present invention includes a method of treating coronary artery disease in a patient or population of patients in need thereof with a bioresorbable polymer stent, wherein the treatment reduces the incidence or severity of angina compared to a metal drug eluting stent. Embodiment fourteen may have one or more, or any combination of the following aspects: the reduced incidence or severity of the angina is observed between 0 and 30 days following PCI, wherein the reduced incidence or severity of the angina is observed between 30 days and 6 months, wherein the reduced incidence or severity of the angina is observed between 6 months and 1 year, the reduced incidence or severity of the angina is sustained beyond 1 year, angina is assessed based on physician diagnosis, wherein the angina is site diagnosed by a physician, the physician diagnosis is recorded on an adverse event form, the angina is assessed based on diagnostic testing, the diagnostic testing is exercise tolerance testing, the diagnostic testing is perfusion imaging, angina is assessed based on the patient(s) reporting of symptoms, the reduced incidence or severity of the angina is assessed by the Seattle Angina Questionnaire, the reduced incidence or severity of the angina is assessed by the Canadian Cardiovascular Society (CCS) Angina Grading Scale, the reduce incidence or severity of the angina leads to lower hospital readmissions compared to that with treatment with the metal drug eluting stent, the reduced incidence or severity of the angina leads to lower diagnostic procedures, the reduced incidence or severity of the angina leads to fewer subsequent angina treatment procedures for the patient(s), the reduced incidence or severity of the angina is caused by improved microvascular resistance, the reduced incidence or severity of the angina is due to less microvascular embolization, the reduced incidence or severity of the angina is caused by increased coronary flow reserve, the reduced incidence or severity of the angina is due to less stress from the scaffold on the vessel wall, the reduced incidence or severity of the angina is due to increases in lumen area, the reduced incidence or severity of the angina is due to increased movement of the vessel, the reduced incidence or severity of the angina is due to less vessel straightening and better vessel conformability of the bioresorbable scaffold.

Embodiment fourteen of the present invention includes a method of reducing total hospital system revascularizations by reducing the number of patients experiencing angina after PCI through use of a bioresorbable stent. The reduced revascularizations are in target lesions and the reduced revascularizations are in non-target lesions.

Embodiment fifteen of the present invention includes a method of treating coronary artery disease (CAD) in a patient in need thereof comprising: selecting a patient in need of treatment of CAD having a lesion in a blood vessel that is an indicator of high risk or a susceptibility of the patient to angina or non-ischemic thoracic chest pain; and implanting a bioresorbable stent at the lesion in a blood vessel of the patient, wherein the implanted scaffold treats the CAD.

Embodiment fifteen may have one or more, or any combination of the following aspects (1)-(10): (1) wherein the lesion is a long diffuse lesion having a length of at least 20 mm; (2) wherein the stent is a bioresorbable polymer stent; (3) wherein the lesion is an ostial lesion; (4) wherein the ostial lesion begins within 1 to 5 mm of an origin of a major epicardial artery; (5) wherein the lesion is a vulnerable plaque suspect lesion; (6) wherein the lesion of aspect (5) has less than 50% occlusion as shown by angiography; (7) wherein the lesion is a bifurcated lesion; (8) wherein the patient experiences no angina or non-ischemic thoracic chest pain for at least 1 year after implantation; (9) wherein the susceptibility comprises a history of angina of the patient within one year prior to implantation; (10) wherein the susceptibility comprises a % diameter stenosis of greater than 70% at a site of implantation of the stent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an exemplary stent scaffold.

FIG. 2A depicts a bioresorbable vascular scaffold (BVS) in a crimped configuration.

FIG. 2B show a cross-selection of a strut of the BVS of FIG. 2A.

FIG. 3 depicts an exemplary stent pattern shown in a planar or flattened view.

FIG. 4A shows event rate data at 2 years for the FAME trial which represents a recent trial using best PCI practices

FIG. 4B depicts the development of post-procedural chest pain (PPCP) according to three treatment groups.

FIG. 5 shows the duration of PPCP after percutaneous coronary intervention.

FIG. 6 depicts the percentage of patients with reported angina through 1 year from ABSORB EXTEND trial and percentage of patients with reported angina for the XIENCE V (XV) drug-eluting stent (DES).

FIG. 7A depicts unadjusted Angina/Angina-Equivalent Kaplan Meier (KM) Curve through 1 year for ABSORB (EXTEND trial) vs. XIENCE V (Spirit IV trial), and Taxus.

FIG. 7B depicts the data for ABSORB and XIENCE from FIG. 7A with differences shown for the time indices and with the Taxus data omitted.

FIG. 7C depicts propensity score-matched Angina KM Curve through 1 year for ABSORB vs. XIENCE V.

FIG. 8 depicts a confocal scanning laser microscopy image of a longitudinal section of a porcine coronary artery stained with a non-specific marker for neural cells 28 days post intervention with a stent.

FIG. 9 depicts a lumen view of the porcine coronary artery of FIG. 8, 28 days post implantation.

FIG. 10 depicts a cut-away section of a blood vessel illustrating arterial mechanical effects of percutaneous coronary intervention (PCI) with a stent.

FIG. 11A depicts ABSORB and XV deployed in curved synthetic blood vessels.

FIG. 11B depicts the average midwall radius of curvature of the deployed ABSORB scaffold and XV stent.

FIG. 11C depicts the normalized excess stress vs. initial radius of curvature at the end of stented segment.

FIG. 11D depicts the normalized extensional stress vs. initial radius of curvature along the axis of stented segment.

FIG. 12A depicts a diagram showing the definition of disease-free segment of an eccentric lesion and the calculation of the percent of disease-free circumference.

FIG. 12B depicts a finite element model of a vessel that includes the adventitia, media, intima, and plaque.

FIG. 12C depicts the finite element model of FIG. 12B with the disease-free arc labeled.

FIG. 12D depicts the finite element model of FIG. 12B with the mean lumen diameter (MLD) and reference vessel diameter (RVD) labeled.

FIGS. 13A-C depict the simulated model for XV, BVS 1.0, and BVS 1.1, respectively, post-deployment.

FIG. 14A compares the circumferential stress of XV, BVS 1.0, and BVS 1.1 in both the media and adventitia at an orientation of 0°.

FIG. 14B compares the circumferential stress of XV, BVS 1.0, and BVS 1.1 and ABSORB in both the media and adventitia at an orientation of 90°.

FIG. 15 depicts the medial layer of the model the simulated deployed XV stent and ABSORB scaffold with level of stress indicated in the elements.

FIG. 16 depicts the medial thickness between struts for ABSORB and XV in a porcine model at 3 days and 28 days post implantation.

FIG. 17 depicts the lumen view of FIG. 9 of the porcine coronary artery, 28 days post-implant.

FIG. 18 depicts the medial thickness under struts for ABSORB and XV in a porcine model at 3 days and 28 days post implantation.

FIG. 19 illustrates reduction of angina for a bioresorbable scaffold throughout is treatment life.

FIG. 20 depicts the percent increased flow rate compared to the stented vessel vs. “a+b” additive vascular restorative theory (VRT) metric for radial fluctuations and positive remodeling.

FIG. 21 depicts the percent increased flow rate compared to the stented vessel vs. “a” for two values of “b,” larger flow rate increase for b=0.04, smaller flow rate increase for b=0.

FIG. 22 depicts the percent increased flow rate compared to the stented vessel vs. “a+b” additive vascular restorative theory (VRT) metric for radial fluctuations and positive remodeling.

FIG. 23 depicts a long diffuse lesion.

FIG. 24 depicts a schematic representation of time dependent behavior of a bioabsorbable scaffold after intervention or deployment.

FIG. 25 depicts in vivo and in vitro data for a bioresorbable vascular scaffold made of poly(L-lactide).

### INCORPORATION BY REFERENCE

All publications and patent applications mentioned in this specification are herein incorporated by reference to the same extent as if each individual publication or patent application was specifically and individually indicated to be incorporated by reference, and as if each said individual publication or patent application was fully set forth, including any figures, herein.

### DETAILED DESCRIPTION OF THE INVENTION

Various embodiments of the present invention include treatment of coronary artery disease (CAD) with bioresorbable stents, in particular bioresorbable polymer stents. The bioresorbable stent can include a support structure in the form of a scaffold made of a material that is bioresorbable, for example, a bioresorbable polymer such as a lactide-based polymer. The scaffold is designed to completely erode away from an implant site after treatment of an artery is completed. The scaffold can further include a drug, such as an antiproliferative, anti-inflammatory , or anti-angina agent. A polymer coating disposed over the scaffold can include the drug which is released from the coating after implantation of the stent. The polymer of the coating may also be bioresorbable.

The method includes positioning the scaffold at a lesion or stenotic segment of an artery and expanding the scaffold at the segment which increase the diameter of the segment. The scaffold maintains patency for a period of time to allow the vessel to remodel at the increase diameter. The scaffold eventually erodes away from the vessel leaving a healed vessel segment.

More specifically, the method involves treatment of CAD with bioresorbable stents with reduced angina or prevention of angina experienced by treated patients or patient populations. The method of treatment may also involve treatment of CAD with bioresorbable stents with reduced angina and non-ischemic chest pain or only reduced ischemic chest pain. The reduced angina or chest pain or reduced degree may be in comparison to that experienced by a patient or populations treated with a metal platform stent.

A “metal platform stent” may refer to a stent having a metallic body or support structure, such as a scaffold. The metallic body may be durable or non-bioerodible when implanted in a body of a patient or mammal. As disclosed herein, a bioabsorbable, bioresorbable, bioerodible stent may have a bioerodible metallic body or support structure.

Patient population may refer to a group or subset of patients treated by a one or more physicians or one or more health care providers. For example, the patient participants of a clinical study may a patient population. The reduced degree can refer to intensity of pain or discomfort, duration of pain or discomfort, frequency of pain, absence or reduction of time periods post implantation. The reduced angina or non-ischemic chest pain or degree thereof may be during 0 to 30 days PI, 30 to 60 days PI, 30 to 180 days PI, 180 days to 1 year PI, 1 to 2 years PI, or any combination thereof. The reduced degree of angina can also refer to a reduced degree of a tested parameter in a diagnostic test for angina, for example, the reduced degree of blood flow or oxygen.

Statement of a time may refer to a time post-implantation or intervention (PI).

The treatment methods relate to the surprising result from clinical studies which show reduction in site diagnosed angina by patients in the ABSORB clinical trial as compared to stents with metallic platforms such as Xience V and Taxus. The reduced reported chest pain, including site diagnosed angina, was found even during post-implantation periods such as the first 30 days before degradation of the polymer of the scaffold affects the mechanical interaction of the scaffold with the vessel.

Angina pectoris, commonly known as angina, is chest pain, discomfort, or pressure in the thoracic region due to ischemia or insufficient supply of blood of the heart muscle, generally due to obstruction or spasm of the coronary arteries. The pain, discomfort, or pressure localized in the chest and is sometimes characterized by a feeling of choking, suffocation, or crushing heaviness. The main cause of angina pectoris is coronary artery disease, due to atherosclerosis of the arteries feeding the heart.

Angina, which is chest pain with an underlying ischemic origin, is to be distinguished from chest pain (chest discomfort, tightness, pain, etc.) occurring in the thoracic region that is of non-ischemic origin. Thus, the term “chest pain” includes both angina and non-ischemic chest pain.

Post-procedural chest pain (PPCP) includes chest pain symptoms reported (usually) in 2-3 weeks following a percutaneous coronary intervention (PCI) procedure.

Stable angina, also known as effort angina, refers to the more common understanding of angina related to myocardial ischemia. Typical presentations of stable angina is that of chest discomfort and associated symptoms precipitated by some activity (running, walking, etc.) with minimal or non-existent symptoms at rest or with administration of sublingual nitroglycerin. Symptoms typically abate several minutes following cessation of precipitating activities and recur when activity resumes. In this way, stable angina may be thought of as being similar to intermittent claudication symptoms.

Worsening (“crescendo”) angina attacks, sudden-onset angina at rest, and angina lasting more than 15 minutes are symptoms of unstable angina (usually grouped with similar conditions as the acute coronary syndrome). Unstable angina is defined as angina pectoris that changes or worsens. It has at least one of these three features: (1) it occurs at rest (or with minimal exertion), usually lasting >10 min; (2) it is severe and of new onset (i.e., within the prior 4-6 weeks); and/or (3) it occurs with a crescendo pattern (i.e., distinctly more severe, prolonged, or frequent than before.

Unstable angina may occur unpredictably at rest which may be a serious indicator of an impending heart attack. What differentiates stable angina from unstable angina (other than symptoms) is the pathophysiology of the atherosclerosis. The pathophysiology of unstable angina is the reduction of coronary flow due to transient platelet aggregation on potentially dysfunctional endothelium, coronary artery spasms, plaque rupture, or coronary thrombosis. The process starts with atherosclerosis, and when inflamed leads to an active plaque, which undergoes thrombosis and results in acute ischemia, which finally leads to myocardial infarction with cell necrosis.

In stable angina, the developing atheroma may be protected with a fibrous cap. This cap over the atherosclerotic plaque may rupture in unstable angina, allowing blood clots to form and further decrease the lumen of the coronary vessel. This explains why unstable angina appears to be independent of activity.

According to the National Institute of Health (http://www.nhlbi.nih.gov/health/health-topics/topics/angina/diagnosis.html), a diagnosis of angina can be performed using a variety of methods or tests. The methods or tests can also be used to determine whether the angina is stable or unstable.

Angina is a primary driver for patients to seek medical attention for a cardiac disorder. Unstable angina, being part of acute coronary syndromes (ACS) is especially worrisome and requires immediate diagnosis and treatment. However, a significant fraction of patients referred to for coronary intervention have stable angina which may be treated by PCI, surgical intervention or optimal medical therapies. The various sites that oversee clinical studies of stents may employ one or any combination of the methods or tests and physician judgement to diagnose angina following a patient report of chest pain.

The clinical data herein discloses angina, a subset of chest pain associated with underlying ischemia- oxygen deprivation to cardiac tissue, specifically site-diagnosed angina (SDA). SDA refers to an adverse event that was diagnosed as angina (stable angina, unstable angina, or angina-equivalent). The data reports only the first SDA event for the patient, regardless of presence/absence of subsequent angina episodes. A diagnosis method includes a patient interview of the patient cardiac history and symptoms. Questions can include: what brings on the pain or discomfort and what relieves it, what does the pain or discomfort feel like (for example, heaviness or tightness), how often does the pain occur, where is the pain or discomfort, how severe is the pain or discomfort, and how long does the pain or discomfort last. A diagnosis may be made based on the interview or diagnostic tests and procedures may be recommended based on interview.

Diagnostic tests include EKG (Electrocardiogram), functional stress testing, coronary angiography and cardiac catheterization, computed tomography angiography, blood tests, and nuclear stress scan.

An EKG test detects and records the heart's electrical activity. An EKG can show signs of heart damage due to coronary heart disease (CHD)/coronary artery disease (CAD) and signs of a previous or current heart attack. However, some patients that have angina can have normal EKGs.

In functional stress testing, a patient exercises to make the heart work hard and beat fast while tests are done. A stress test can show possible signs and symptoms of CHD. As part of some stress tests, pictures are taken of the heart during the exercise and while at rest. These imaging stress tests can show how well blood is flowing in various parts of the heart and how well the heart pumps blood when it beats.

A chest x-ray takes pictures of the organs and structures inside the patient's chest, such as heart, lungs, and blood vessels. A chest x-ray can reveal signs of heart failure and signs of lung disorders and other causes of symptoms not related to CHD. However, a chest x-ray alone is not enough to diagnose angina or CHD.

Coronary angiography and cardiac catheterization uses dye and special x rays to visualize the flow passing through coronary arteries. In this procedure, a catheter is put into a blood vessel and threaded into coronary arteries, and the dye is released into the bloodstream. X-rays are taken while the dye is flowing through the coronary arteries.

Computed tomography uses dye and x rays to show blood flow through the coronary arteries. Dye is injected through an IV line during a scan. While inside a CT scanner, an x-ray tube moves the patient's body to take pictures of different parts of your heart and a computer puts the pictures together to make a three-dimensional (3D) picture of the whole heart.

Blood tests check the levels of cardiac enzymes indicative of heart muscle injury (e.g., creatine kinase myocardial band—CKMB). Blood tests reveal levels of certain fats, cholesterol, sugar, and proteins in the blood. Abnormal levels may show that risk factors for CAD.

A nuclear scan (e.g., myocardial perfusion, thallium, gamma scintigraphy, or positron emission tomography (PET)) is an imaging test that uses a radioactive substance called a tracer to look for disease or poor blood flow in the heart.

Although the surprising results are for the ABSORB cohort B scaffold, the hypothesized mechanism for the reduction in angina indicates that such results may apply generally to bioresorbable polymer scaffolds, and more generally to polymer scaffolds. Even more generally, the surprising results would apply to scaffolds composed of composites of polymers and metallic or ceramic materials that exhibit the disclosed scaffold-vessel interactions responsible for reduction or prevention of angina.

A method of treating coronary artery disease (CAD) in a patient or population of patients may include identifying a patient or population of patients in need of treatment of CAD. The bioresorbable stent may be implanted in the patient or population of patients for treating CAD and the stent may be implanted at a lesion or stenotic portion of a blood vessel in the patient or population of patients. The implanted stent may treat the CAD and the patient or population of patients may experience no angina or non-ischemic thoracic chest pain or a reduced degree of angina non-ischemic thoracic chest pain as compared to a metallic stent.

Reduced angina or reduced degree of angina may refer to a bioresorbable stent as compared to a metal platform stent. The reduced angina may correspond to a comparison of a patient specific angina event to statistical data of metal platform stents. The reduced angina may correspond to a comparison of statistical data on the patient population to statistical data of metal platform stents. Examples of these comparisons are provided herein. The type (e.g., tradename) or design of bioresorbable stent may have been shown in clinical trials to have lower rate of angina, for example, lower site diagnosed angina, than the rate of angina of one or more metal platform stents, for example, XIENCE V or Taxus, shown in clinical trials. The rate of angina may be expressed in terms of one or more standard known in the field of clinical trial analysis.

The interaction of the scaffold with the vessel may be such that the patient experiences no chest pain during the first 30 days post implantation (PI), 30 to 60 days PI, 60 to 180 days PI, or 180 to 300 days.

The method of treatment may further include identifying a patient or population of patients that are susceptible to angina or non-ischemic chest pain. The method of treatment may include identifying a characteristic of patients in need of treatment for CAD which makes the patients susceptible to angina or identifying factors, conditions, characteristics in patients in need of treatment for CAD which makes patients susceptible to angina.

In other embodiments, the method may include recommending treatment or describing advantages relating to reduced angina as compared to metal platform stents with a bioresorbable stent for treating the CAD of patients. The recommendation may be based on factors, conditions, characteristics being present in a patient or patient population. The method includes implanting the bioresorbable polymer stent for treating CAD in such patients based on such recommendations with reduced or absence of angina in the patient. The recommendation or selection may be indicated as an alternative over a metallic platform stent and may include indicating the reduction or prevention of angina as an advantage over the metallic platform stent.

The recommendations or descriptions of advantages may be based on performance (e.g., in clinical trials) of the type or design recommended or be based on the performance (e.g., clinical trials) of another type or design of bioresorbable stent.

Factors, conditions, or characteristics that are indicators of susceptibility to angina include type of coronary lesion, suffering from a non-CAD disease, race, ethnicity, or gender. Susceptibility to angina refers susceptibility to angina before, after, or both before and after stent implantation.

Susceptibility of patients to angina of a selected race, ethnicity, or gender may be demonstrated or indicated by published or unpublished clinical data, standards of care sanctioned by local, state, or national, governments, insurance companies, medical associations, a medical professional, or a medical device statement or publication in print or on the internet. For example, it is known that patients of Indian sub-continent descent are susceptible to angina. Susceptibility of patients to angina having a non-CAD disease may be demonstrated or indicated by published or unpublished clinical data, standards of care sanctioned by local, state, or national, governments, insurance companies, medical associations, a medical professional, or a medical device statement or publication in print or on the internet. For example, it is recognized that patients suffering diabetes, obesity, or prone to vasospasm are susceptible. Male patients, due to differences in presentation of symptoms between males and females, may be susceptible to angina.

Susceptibility to angina after implantation may correspond to patient history of angina prior implantation of a bioabsorbable stent. The patient history of angina may correspond to symptoms of angina within a certain period prior to implantation, for example, within 2 years, within 1 year, within 6 months, or within a month of implantation. The angina history may be symptomatic, stable, or unstable.

Susceptibility to angina after implantation may correspond to a degree stenosis of an implant site of an artery to be treated prior to implantation of a stent at the site. For example, a patient may have a greater susceptibility to angina after implantation if the % diameter stenosis (DS) prior to implantation is greater than 50%, greater than 60%, greater than 70%, greater than 80%, 50 to 80%, 50 to 70%, 60 to 70%, or 70 to 80%. % “Diameter stenosis” (% DS) is the percent difference between the reference vessel diameter (RVD) and the minimal lumen diameter (MLD): (RVD-MLD)/RVD. “Reference vessel diameter” (RVD) is the diameter of a vessel in areas adjacent to a diseased section of a vessel that appear either normal or only minimally diseased. “Minimal lumen diameter” (MLD) is the diameter of a diseased section of a vessel at the site of maximal reduction in the diameter. The DS, RVD, and MLD may be measured by angiography.

Susceptibility of patients to angina having certain types of coronary lesions or vessels to be treated by the scaffold may be demonstrated by published or unpublished clinical data, standards of care sanctioned by local, state, or national, governments, insurance companies, or medical associations. For example, it is known that patients having long diffuse lesions, ostial lesion, vulnerable plaque suspect lesion (less than 50% occlusion by angiography), or bifurcated lesions are susceptible to angina.

Additionally, it is known that patients having small vessels (2 to 3 mm reference vessel diameter) are susceptible to angina. “Reference vessel diameter” (RVD) is the diameter of a vessel in areas adjacent to a diseased section of a vessel that appear either normal or only minimally diseased.

An ostial lesion may refer to a lesion which begins within 3-5 mm of the origin of a major epicardial artery.

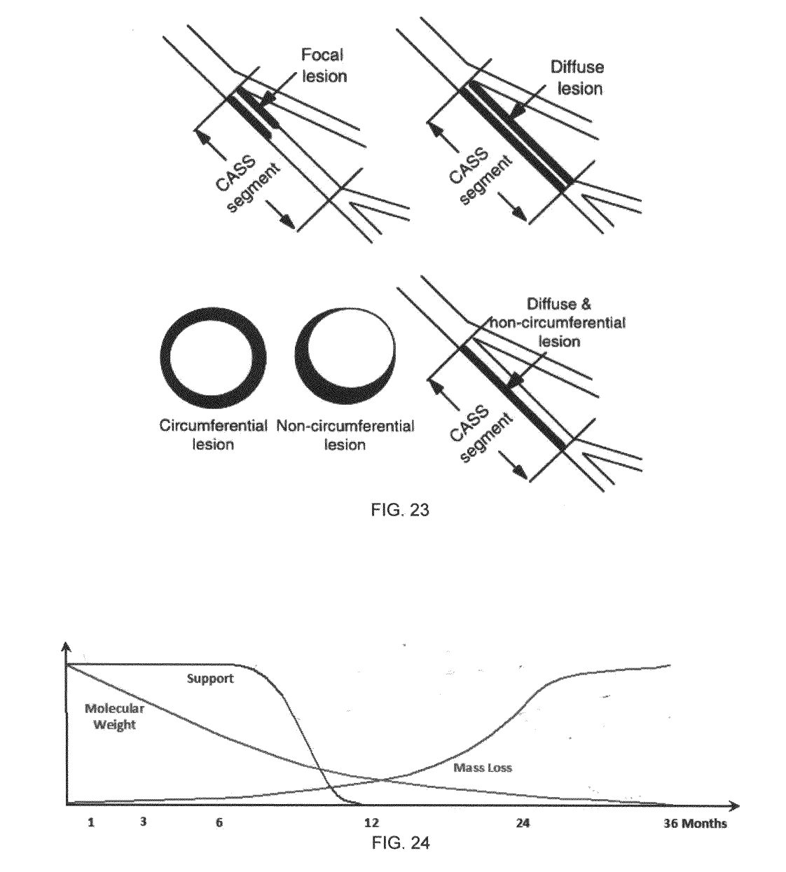

Long diffuse lesion may be defined, as shown in FIG. 23, as a lesion greater than 20 mm or that extends longitudinally throughout an entire length of coronary artery surgery study segment. In other words, if part of a segment is normal, the lesion is defined as focal. Focality thus describes longitudinal abruptness of a lesion. On the other hand, circumferentiality depicts the circumferential distribution of a lesion. As shown, a lesion involving the entire circumference of the vessel is defined as circumferential. Therefore, distribution of a lesion is described by 2 terms, focal and circumferential.

Bifurcated or bifurcation lesion may refer to a coronary artery narrowing that may involve the proximal main vessel, the distal main vessel, and the side branch.

It is further believed that a patient is susceptible to angina in the treatment of in-stent restenosis (ISR) with repeat-procedures like stenting in-stent restenosis (ISR) lesions. It is likely that any deep wall trauma and nerve stimulation would be exacerbated by the presence of extra metallic struts.

ISR refers to restenosis at a stented segment which leads to ISR lesions. ISR can be defined clinically or angiographically. Clinically it is defined as the presentation of recurrent angina or objective evidence of myocardial ischaemia. Angiographic ISR is the presence of greater than 50% diameter stenosis in the stented segment. ISR has been classified based on the length of the lesion, as focal (<10 mm) or diffuse (>10 mm).

Treatment of an ISR lesion with a bioabsorbable stent may result in reduced post-procedural non-ischemic chest pain. The benefits observed with treatment may be amplified in treatment of ISR. The benefit would, for example, exist in cases of overlapping stent deployment, in which metallic struts may lead to an exacerbated incidence of focal angina. More flexible and absorbable platforms may provide reduced chronic stretch pain /vascular stress, circumferential, and potentially bending.

The treatment method further includes a step of selecting a bioresorbable polymer stent having a reduced risk of inducing angina in the patient. The method then includes implanting the stent in the patient resulting in reduced or no angina or non-ischemic chest pain. The reduction or absence of angina may be due to the mechanisms relating to scaffold-vessel interactions disclosed herein. The method then includes implanting the stent in the patient resulting in a reduction of or no post-procedural chest pain.

In addition to the step of identifying factors, conditions, or characteristics or the step of recommending or describing advantages of treatment with a bioresorbable stent, the method of treatment may include additional features. In a further embodiment, the method of treatment may include providing or sending the bioresorbable stent to a medical facility, medical professional, or distributer for distribution to a medical facility or medical professional for treatment of a patient or population of patients in need of treatment of the CAD that has or does not have one or more of the factors, conditions, or characteristics. In an embodiment, the method may include providing a plurality of the recommended bioresorbable stents to a health care provider that implants the stents in a population of patients and a statistically significant number of the population of patients experiences lower rate of angina than has been shown for a metal platform stent. In another embodiment, the recommended bioresorbable stent has been shown to provide a reduced rate of angina as compared to a metal platform stent in other populations, for example, in preclinical or clinical studies. The angina reduction can be based on any of the diagnosis methods disclosed herein and can be based on SDA.

Identifying factors, conditions, or characteristics can take the form of any oral, electronic, printed, or telephonic communications. Examples include oral communications at business meetings, any type of communication at technical meetings; marketing or promotional documents in print or on an internet website; email communications; telephone communication; webinars, and seminars and other educational situations.

Recommending or describing advantages can take the form of any oral, electronic, printed, or telephonic communications. Examples include oral communications at business meetings, any type of communication at technical meetings; marketing or promotional documents in print or on an internet website; email communications; telephone communication; webinars, and seminars and other educational situations.

The present invention is applicable to, but is not limited to, self-expandable stents, balloon-expandable stents, stent-grafts, and generally tubular medical devices in the treatment of artery disease. The present invention is further applicable to various stent designs including wire structures and woven mesh structures.

Self expandable or self expanding stents include a bioabsorbable polymer scaffold that expands to the target diameter upon removal of an external constraint. The self expanding scaffold returns to a baseline configuration (diameter) when an external constraint is removed. This external constraint could be applied with a sheath that is oriented over a compressed scaffold. The sheath is applied to the scaffold after the scaffold has been compressed by a crimping process. After the stent is positioned at the implant site, the sheath may be retracted by a mechanism that is available at the end of the catheter system and is operable by the physician. The self expanding bioabsorbable scaffold property is achieved by imposing elastic deformation to the scaffold during the manufacturing step that compresses the scaffold into the sheath.

The bioabsorbable scaffold may also be expanded by a balloon. In this embodiment the scaffold is plastically deformed during the manufacturing process to tightly compress the scaffold onto a balloon on a catheter system. The scaffold is deployed at the treatment site by inflation of the balloon. The balloon will induce areas of plastic stress in the bioabsorbable material to cause the scaffold to achieve and maintain the appropriate diameter on deployment.

A stent scaffold can include a plurality of cylindrical rings connected or coupled with linking elements. For example, the rings may have an undulating sinusoidal structure. When deployed in a section of a vessel, the cylindrical rings are load bearing and support the vessel wall at an expanded diameter or a diameter range due to cyclical forces in the vessel. Load bearing refers to the supporting of the load imposed by radial inwardly directed forces. Structural elements, such as the linking elements or struts, are generally non-load bearing, serving to maintain connectivity between the rings. For example, a stent may include a scaffold composed of a pattern or network of interconnecting structural elements or struts.

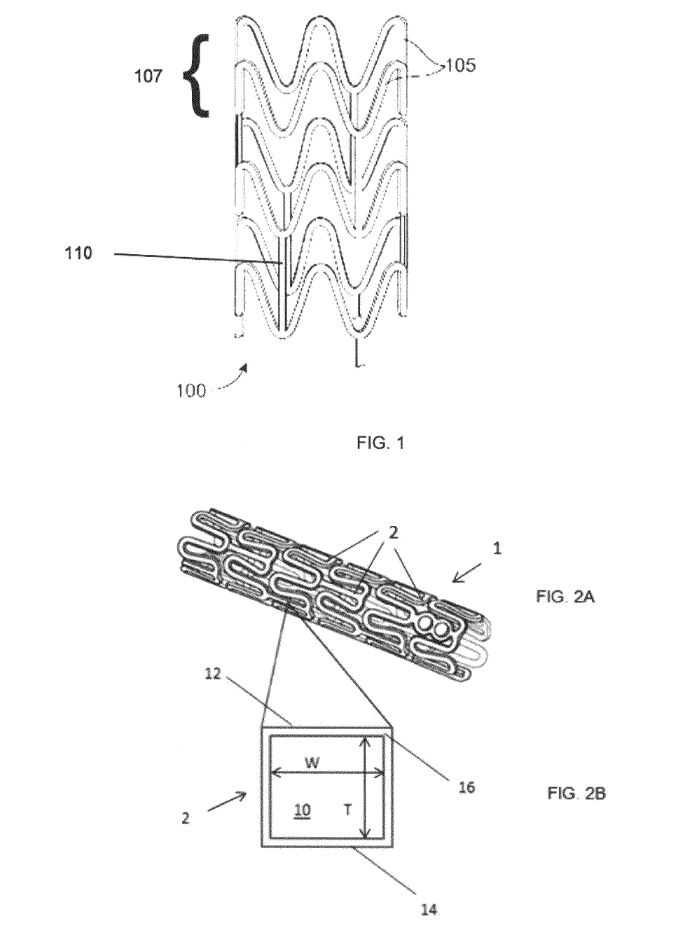

FIG. 1 depicts a view of an exemplary stent 100. In some embodiments, a stent may include a body, backbone, or scaffold having a pattern or network of interconnecting structural elements 105. Stent 100 may be formed from a tube (not shown). FIG. 1 illustrates features that are typical to many stent patterns including undulating sinusoidal cylindrical rings 107 connected by linking elements 110. The cylindrical rings are load bearing in that they provide radially directed force to support the walls of a vessel. The linking elements generally function to hold the cylindrical rings together. A structure such as stent 100 having a plurality of structural elements may be referred to as a stent scaffold or scaffold. Although the scaffold may further include a coating, it is the scaffold structure that is the load bearing structure that is responsible for supporting lumen walls once the scaffold is expanded in a lumen.

The structural pattern in FIG. 1 is merely exemplary and serves to illustrate the basic structure and features of a stent pattern. A stent such as stent 100 may be fabricated from a polymeric tube or a sheet by rolling and bonding the sheet to form the tube. A tube or sheet can be formed by extrusion or injection molding. A stent pattern, such as the one pictured in FIG. 1, can be formed on a tube or sheet with a technique such as laser cutting or chemical etching. The stent can then be crimped on to a balloon or catheter for delivery into a bodily lumen. Alternatively, the scaffold design may be composed of radial bands that slide to increase the diameter of the scaffold. Such a design utilizes a locking mechanism to fix the stent at a target diameter and to achieve the final radial strength. In other embodiments, the scaffold design could be braided polymer filaments or fibers.

The treatment methods disclosed herein can apply to bioresorbable scaffolds for both coronary and peripheral treatment. Bioresorbable polymer scaffolds for coronary artery treatment can have a length between 8 to 48 mm. Such coronary scaffolds may be laser cut from polymer tubes with a diameter between 2.0 mm to 5.5 mm and with a thickness/width of 80-160 microns.

The coronary scaffold may be configured for being deployed by a non-compliant or semi-compliant balloon from about a 1.1 to 1.5 mm diameter (e.g., 1.35 mm) crimped profile. Exemplary balloon sizes include 2.5 mm, 3.0 mm, 3.5 mm, 4.0 and 4.5 mm, where the balloon size refers to a nominal inflated diameter of the balloon. The scaffold may be deployed to a diameter of between 2.5 mm and 5 mm, 2.5 to 4.5 mm, or any value between and including the endpoints. The pressure of the balloon to deploy the scaffold may be 7 to 30 psi. Embodiments of the invention include the scaffold in at a crimped diameter over and in contact with a deflated catheter balloon.

The intended deployment diameter may correspond to, but is not limited to, the nominal deployment diameter of a catheter balloon which is configured to expand the scaffold. The balloon pressure and the diameter to which the balloon inflates and expands the scaffold may vary from deployment to deployment. For example, the balloon may expand the scaffold in a range between the nominal inflated diameter to the nominal inflated diameter plus 0.5 mm, e.g., a 3.0 mm balloon may expand a scaffold between 3 and 3.5 mm. In any case, the inflated diameter at deployment is less than the rated burst diameter of the balloon.

A scaffold may be laser cut from a tube (i.e., a pre-cut tube) that is greater than or . less than an intended deployment diameter. In this case, the pre-cut tube diameter may be 0.5 to 1.5 times the intended deployment diameter or any value or range in between and including the endpoints.

Compared with bare metal stents, drug-eluting stents (DES) that are not bioresorbable have been shown to be safe and to result in greater absolute reductions in target lesion revascularization (TLR) and target vessel revascularization. A DES refers to a stent including a support structure (e.g., scaffold) and also includes a drug-eluting coating over the support structure. The coating can include a polymer and a drug. The polymer functions as a drug reservoir for delivery of the drug to a vessel. The polymer can be non-biodegradable or bioresorbable. The support structure may be bioresorbable, such as a bioresorbable polymer or bioerodible metal, however, the term “DES” typically is used to refer to stents having a durable or non-degradable metal support structure with a drug-eluting coating.

The ABSORB Bioresorbable everolimus eluting vascular scaffold (ABSORB BVS) of Abbott Vascular Inc. of Santa Clara, CA was recently developed to provide an approach to treating coronary artery lesions with transient vessel support and drug delivery. Preclinical evaluation in an animal model demonstrated substantial polymer degradation at 2-years post ABSORB BVS implantation, with complete disappearance of the BVS strut “footprint” in the vessel wall within a 3-4 year period. The first generation BVS (BVS revision 1.0) was tested in the ABSORB cohort A trial and demonstrated promising results with a low event clinical rate at up to 4 years follow up (EuroIntervention 2012;7:1060-1061). The device was however limited by a slightly higher acute recoil compared to conventional metallic platform stents.

Improvements in design were therefore introduced in the second generation BVS (BVS revision 1.1), notably an enhanced mechanical strength, more durable support to the vessel wall, a reduced maximum circular unsupported surface area and a more uniform strut distribution and drug delivery. The performance of the next generation BVS revision 1.1 was subsequently investigated in the ABSORB Cohort B Trial which reported excellent clinical results at 1, 2, and 3 year follow-up (J Am Coll Cardio1.2011; 58: B66; Circulation. 2009;120:S951).

The polymer backbone is made of poly(L-lactide). The nominal inner diameter of the scaffold is 3 mm and the length is 18 mm. The struts have a width of about 165 microns and thickness of about 152 microns. The coating is a mixture of poly(DL-lactide) and everolimus at a 1:1 (w/w) ratio of polymer to drug. The coating is about 1 to 3 microns in thickness. The drug dose density is 100 μg/cm², which is the drug mass per unit scaffold surface area. The surface area of the 3.0×18mm scaffold is 160 mm², so the target drug dose is about 160 μg. The surface area of the scaffold per unit scaffold length is about 8.9 mm²/mm. The scaffold artery ratio (SAR) for the stent is approximately 24%, based on a square strut cross-section. As a comparison, the SAR for XIENCE V (3mm×18 mm) is 13.7%. Interventional Cardiology, 2011; 6(2):134-141.

FIG. 2A depicts a bioresorbable vascular scaffold (BVS) 1 composed of a plurality of struts 2 in a crimped configuration. FIG. 2B show a cross-selection of a strut 2 showing the polymer scaffold body, polymer backbone, or core of the strut surrounded by a drug/polymer coating or matrix 16. The cross-section of the strut has an abluminal surface or side 12 that faces the vessel wall and a luminal surface or side 14 that faces the lumen of the vessel. The strut cross-section shown is rectangular with a width (W) and thickness (T). The scaffold cross-section may be approximately square with an aspect ratio T/W close to 1.

In a preferred embodiment a scaffold for coronary applications has the stent pattern described in U.S. application Ser. No. 12/447,758 (US 2010/0004735) to Yang & Jow, et al. Other examples of stent patterns suitable for PLLA are found in US 2008/0275537. FIG. 3 depicts exemplary stent pattern 700 from US 2008/0275537. The stent pattern 700 is shown in a planar or flattened view for ease of illustration and clarity, although the stent pattern.700 on a stent actually extends around the stent so that line A-A is parallel or substantially parallel to the central axis of the stent. The pattern 700 is illustrated with a bottom edge 708 and a top edge 710. On a stent, the bottom edge 708 meets the top edge 710 so that line B-B forms a circle around the stent. In this way, the stent pattern 700 forms sinusoidal hoops or rings 712 that include a group of struts arranged circumferentially. The rings 712 include a series of crests 707 and troughs 709 that alternate with each other. The sinusoidal variation of the rings 712 occurs primarily in the axial direction, not in the radial direction. That is, all points on the outer surface of each ring 712 are at the same or substantially the same radial distance away from the central axis of the stent.

The stent pattern 700 includes various struts 702 oriented in different directions and gaps 703 between the struts. Each gap 703 and the struts 702 immediately surrounding the gap 703 define a closed cell 704. At the proximal and distal ends of the stent, a strut 706 includes depressions, blind holes, or through holes adapted to hold a radiopaque marker that allows the position of the stent inside of a patient to be determined.

One of the cells 704 is shown with cross-hatch lines to illustrate the shape and size of the cells. In the illustrated embodiment, all the cells 704 have the same size and shape. In other embodiments, the cells 704 may vary in shape and size. Still referring to FIG. 3, the rings 712 are connected to each other by another group of struts that have individual lengthwise axes 713 parallel or substantially parallel to line A-A. The rings 712 are capable of being collapsed to a smaller diameter during crimping and expanded to their original diameter or to a larger diameter during deployment in a vessel. Specifically, pattern 700 includes a plurality of hinge elements at the crests and troughs. When the diameter of a stent having stent patter 700 is reduced or crimped, the angles at the hinge elements decrease which allow the diameter to decrease. The decrease in the angles results in a decrease in the surface area of the gaps 703. In general, for most coronary applications, the diameter of the scaffold is 2 to 5 mm, or more narrowly 2.5 to 4.0 mm. In general, the length of the scaffold is 8 to 48 mm, or more narrowly, 8 to 12 mm, 12 to 18 mm, 18 mm to 38 mm. The scaffold for may be configured for being deployed by a non-compliant balloon, e.g., 2.5 to 4.5 mm diameter, from about a 1.8 to 2.2 mm diameter (e.g., 2 mm) crimped profile. The coronary scaffold may be deployed to a diameter of between about 2.5 mm and 4.5 mm.

The present application includes results and analysis from the ABSORB EXTEND Trial. The ABSORB EXTEND study is a single-arm trial evaluating Absorb in patients with more complex heart disease. Details of the trial can be found at http://clinicaltrials.gov/ct2/show/ NCT01023789. Clinical results for the ABSORB stent are compared to that of a metallic stent, specifically, XIENCE V® stent. XIENCE V has a cobalt chromium backbone structure with a biostable drug-eluting coating.

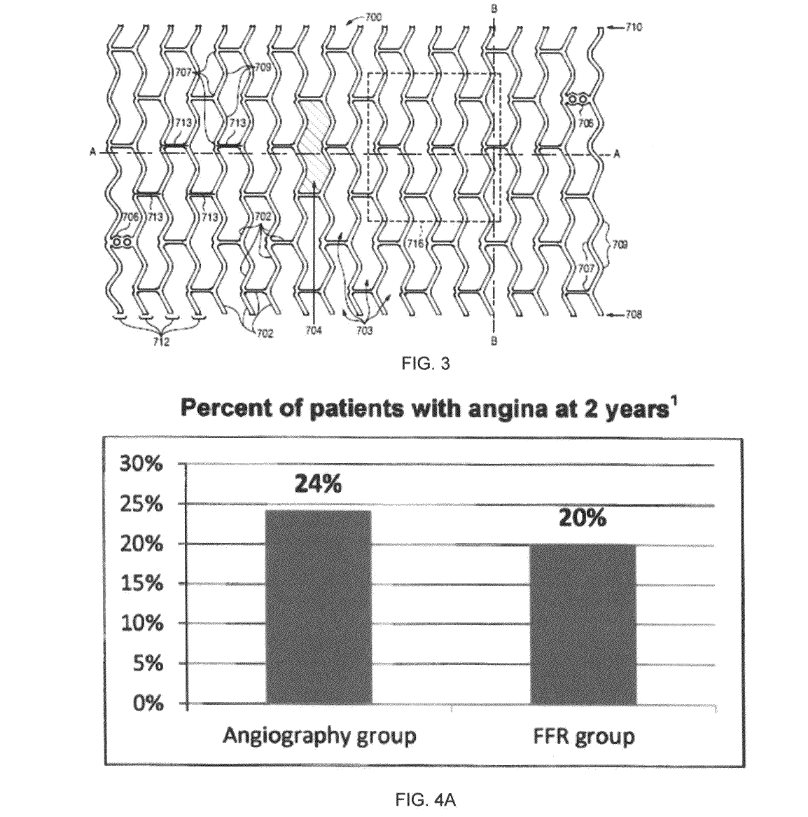

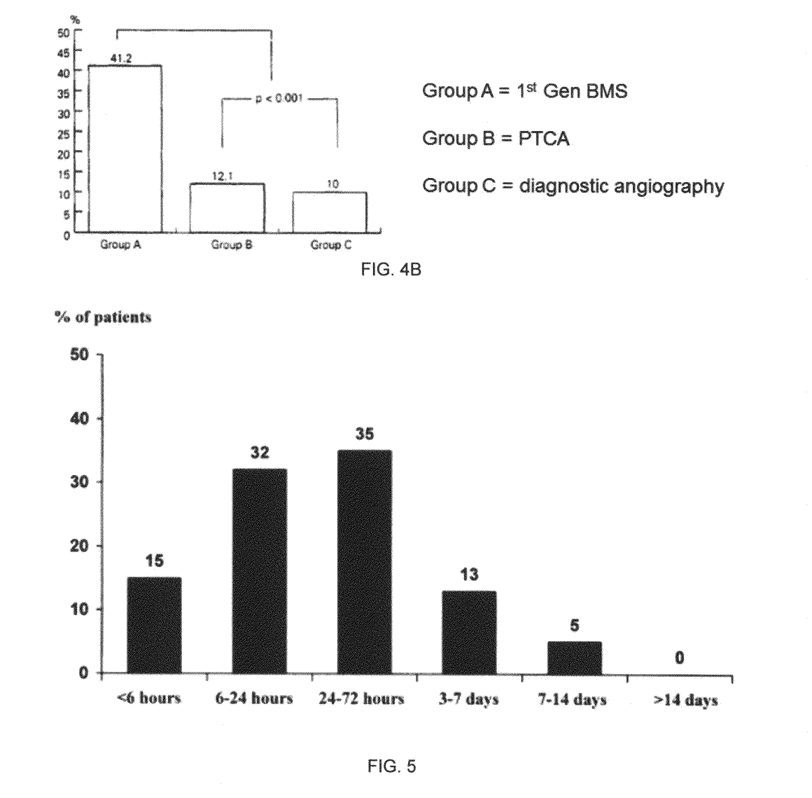

PCI is minimally invasive compared to surgery and will address stable angina caused by ischemia. Clinical data indicate that post-procedural chest pain is induced by PCI including diagnostic angiography, percutaneous transluminal coronary angioplasty (PTCA), and stent implantation. While PCI does ameliorate angina, there is still room for further improvement. FIG. 4A shows event rate data at 2 years for the FAME trial which represents a recent trial using best PCI practices. Pijls, N et al; Journal of the American College of Cardiology; Vol 5, No 3, 2010; ISSN: 0735-1097. Table 1A, below, summarizes data of the FAME trial. These data are not the percentage of patients at two years which have angina but represents the cumulative angina rate at two years.

TABLE 1A
Summary of data of the FAME trial.

| | Angiography | | | RR With FFR |
|---|---|---|---|---|
| | Group | FFR Group | | Guidance |
| | (n = 496) | (n = 509) | p Value | (95% CI) |
| Events at 2 Years | | | | |
| Total number of events | 142 | 106 | | |
| Number of events per patient | 0.29 ± 0.60 | 0.21 ± 0.48 | 0.17 | |
| Death | 19 (3.8) | 13 (2.6) | 0.26 | 0.67 (0.33-1.34) |
| Mycardial Infarction | 49 (9.9) | 31 (6.1)) | 0.03 | 0.62 (0.40-0.96) |
| CABG or repeat PCI | 63 (12.7) | 64 (10.6) | 0.30 | 0.84 (0.59-1.18) |
| Death or myocardial Infarction | 64 (12.9) | 43 (8.4) | 0.02 | 0.66 (0.45-0.94) |
| Death, myocardial Infarction, CABG or repeat | 111 (22.4) | 91 (17.9) | 0.08 | 0.80 (0.62-1.02) |
| PCI | | | | |
| Functional status at 2 yrs | | | | |
| Patients without event and free from | 264 (64.8) | 315 (68.2) | 0.29 | |
| angina† | | | | |
| Patients free from angina† | 332 (75.8) | 369 (79.9) | 0.14 | |
| Number of anti-anginal medications ± | 1.2 ± 0.8 | 1.2 ± 0.7 | 0.68 | |

FIG. 4B depicts the development of post-procedural chest pain (PPCP) according to three treatment groups. Group A (n=21) is bare metal stent (BMS) intervention. Group B (n=4) is PTCA. Group C (n=6) is diagnostic angiography. Jeremias, A et al. Herz 24: 126-131, 1999. The highest incidence of chest pain is with BMS intervention with 41.2% of patients experiencing PPCP. Group B and C patients experience PPCP at 12.1% and 10%.

Not all chest pain is ischemic angina. Some chest pain is musculoskeletal or GI induced. Chest pain after successful coronary intervention is a common problem. In the acute timeframe, this pain could be due to arterial stretch injury, vessel dissection, acute coronary artery closure, coronary artery spasm, myocardial infarction, or simply coronary artery trauma. The adventitia contains nerves and pain can be induced by local, persistent vessel stretch or deep adventitial injury. Kini Am Coll Cardiol, 41, 1, 2003, p. 33; Jeremais A, Herz 1999;24:126-131.

PPCP is experienced for a duration of at least hours, days, and weeks post-intervention. FIG. 5 shows the duration of PPCP after percutaneous coronary intervention. Kini K S et al. JACC 41: 33-38, 2003. The duration of post-procedural chest pain after stenting is known to subside after roughly two weeks as depicted in FIG. 5. PPCP is seen to increase from a period of <6 hrs to peak at 24-72 hrs and decrease after this period to 0 at >14 days.

An important question is whether the unique characteristics and benefits of a bioresorbable coronary scaffold such as ABSORB can be used to enhance PCI, specifically with respect reducing incidence, duration, and degree of angina or PPCP. The benefits include improvement of patient quality of life due to reductions in angina. There are also reduced economic benefits derived from lower readmissions and less healthcare system utilization.

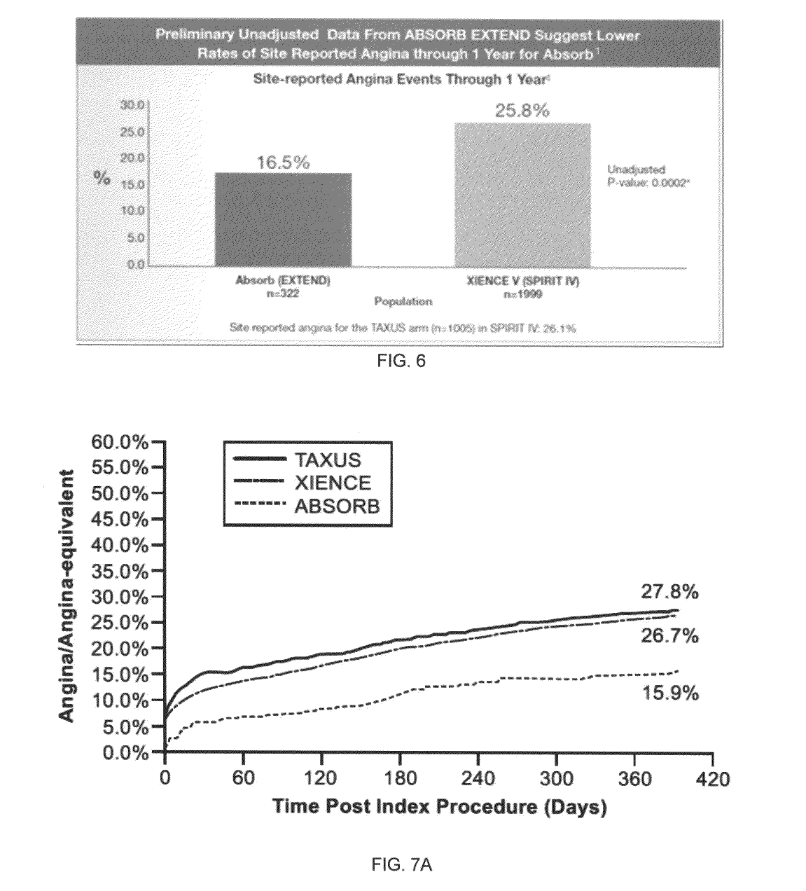

Surprising results from the ABSORB EXTEND clinical study showed lower site diagnosed angina (SDA) as compared to polymer coated metal platform stent DES). Whether PPCP, angina, or SDA, reductions of such events can potentially reduce the economic burden associated with health care of CAD patients. Table 1B shows adjusted and unadjusted SDA for ABSORB EXTEND compared to Xience V in SPIRIT IV Trial. FIG. 6 depicts the percentage of patients with site diagnosed angina through 1 year from ABSORB EXTEND and percentage of patients with reported angina for the XIENCE V (XV) DES from SPIRIT IV. The percentage of patients with site diagnosed angina with ABSORB is 16.5%. This is significantly less than reported angina for XIENCE V which is 25.8% of patients. The ABSORB data is from A. Bartorelli, Most Recent Findings From ABSORB Clinical Trial Programme, EuroPCR 2013. More recent, not yet reported data, is 16% site diagnosed angina for ABSORB and 27.9% site diagnosed data for XIENCE V. The ABSORB EXTEND data is non-randomized data for hypothesis generation and analysis is not propensity adjusted. The data for the SDA was collected on AE (adverse event) electronic case report forms. Populations for ABSORB EXTEND include the following geographies: EMEA, CALA, APAC (excluding non-Japanese Asians). The XIENCE data is from the SPIRIT IV clinical study and was conducted in US.

The unadjusted propensity score matched site diagnosed angina at 1 year follow-up for ABSORB, and Xience V in SPIRIT IV Trial. The adjusted score shows an even larger difference in between the ABSORB and XV reported angina, 16.0% vs. 27.9%, a difference of 11.9%.

TABLE 1B
Adjusted and unadjusted SDA for ABSORB EXTEND compared to
Xience V in SPIRIT IV Trial.

| | | BVS* | XV |
|---|---|---|---|
| | | (EXT excl.) | SPIRIT IV |
| | Follow-up | non-JPN Asians) | Non-Complex |
| Unadjusted | 1-Yr | 15.9% (60/378) | 27.1% (542/2000) |
| Propensity Score | 1 Yr | 16.0% (46/287) | 27.9% (168/602) |
| Adjusted | | | |

*Based on angina/angina-equivalent

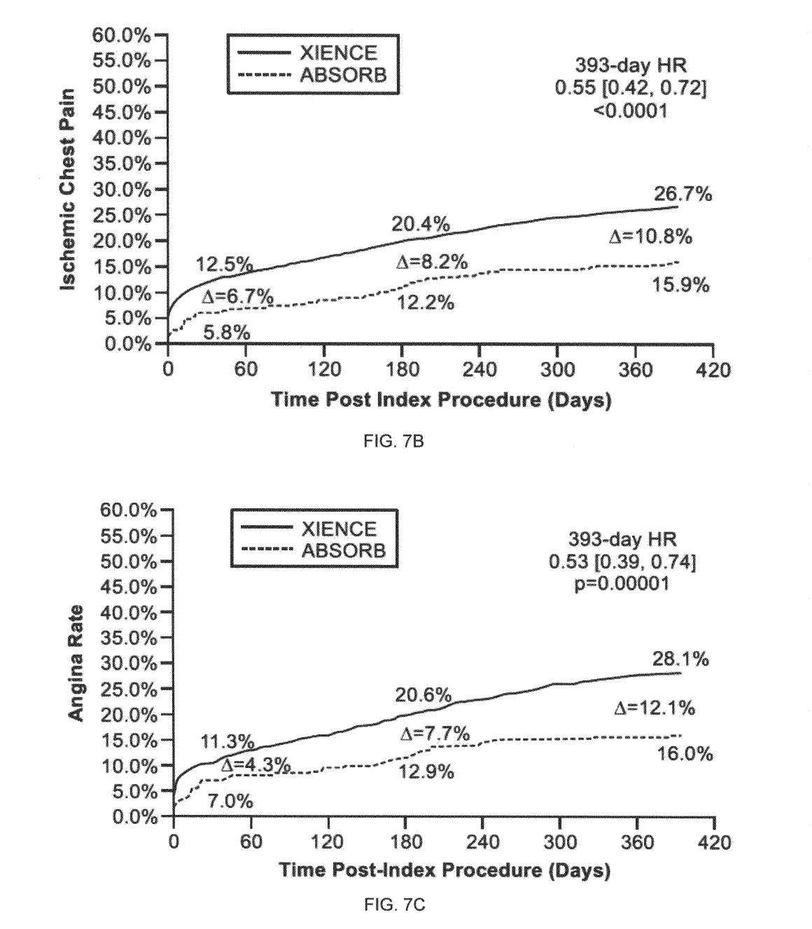

FIGS. 7A-7C depicts cumulative distribution curves of angina after PCI. FIG. 7A depicts unadjusted Angina/Angina-Equivalent KM Curve through 1 year for ABSORB (EXTEND trial) vs. XIENCE V (Spirit IV trial), and Taxus. FIG. 7B depicts the data for ABSORB and XIENCE from FIG. 7A with differences shown for the time indices and with the Taxus data omitted. FIG. 7C depicts propensity score-matched Angina KM Curve through 1 year for ABSORB vs. XIENCE V. These curves show both an acute phase for angina events extending out to 30 days, and then a more steady rate of angina events out to the one year time point. This data encompasses site diagnosed angina and whether the angina was mild or severe, it was classified equally as an angina event. Also, it was not differentiated whether a patient had one or more angina events; a single angina event added them to the cumulative curve.

The propensity score matched analysis balances the baseline characteristics of both clinical trials to ensure that the treatment effect is not due to baseline differences. Like XV, Taxus is a DES made of metal with a polymer and drug coating.

Table 2A shows the number of patients in the studies for each stent at intervention and three time points. As shown by FIG. 7A, the percentage of patients with reported angina is significantly less than both the XV and Taxus DES stents from intervention to 393 days. The reported angina for XV and Taxus are substantially the same throughout the period (i.e., within about 1 to 5%).

TABLE 2A
Unadjusted data for number of patients at risk in the ABSORB, XV, and
Taxus clinical studies from intervention and 37, 194, and 393 days post
intervention with SDA events for each time index.

Time post-Index
Procedure (days)

| | 0 | 37 | 194 | 393 |
|---|---|---|---|---|
| Absorb Subjects At Risk: | 378 | 355 | 330 | 314 |
| # Events | 5 | 22 | 46 | 60 |
| XIENCE Subjects At Risk: | 2051 | 1784 | 1600 | 1438 |
| # Events | 114 | 256 | 415 | 542 |
| TAXUS Subjects At Risk: | 1032 | 859 | 782 | 711 |
| # Events | 65 | 160 | 228 | 283 |

TABLE 2B
Propensity score-matched data for number of patients at risk in the
ABSORB, XV, and Taxus clinical studies from intervention and 37, 194,
and 393 days post intervention with SDA events for each time index.

Time post-Index
Procedure (days)

| | 0 | 37 | 194 | 393 |
|---|---|---|---|---|
| Absorb Subjects At Risk: | 378 | 355 | 330 | 314 |
| # Events | 5 | 22 | 46 | 60 |
| XIENCE Subjects At Risk: | 2051 | 1784 | 1600 | 1438 |
| # Events | 114 | 256 | 415 | 542 |
| TAXUS Subjects At Risk: | 1032 | 859 | 782 | 711 |
| # Events | 65 | 160 | 228 | 283 |

With respect to the various embodiments of the present invention, a bioresorbable stent may have shown an SDA in patient populations of less than 8%, less than 6%, or 4% to 8% at 37 days PI, 30 days, or 30 to 40 days PI. The bioresorbable stent may have shown an SDA in patient populations of less than 12%, less than 14%, or 10% to 14% at 193 days PI, 180 days PI, or 175 to 195 days PI. The bioresorbable stent may have shown an SDA in patient populations of less than 16%, less than 18%. 14% to 20% at 393 days PI, 1 year PI, or 365 to 400 days PI.

“Shown” can refer to unpublished, published, non-publicly known, or publicly known information or data.

The bioresorbable stent may have a backbone, body, or scaffold that is PLLA-based, made of PLLA, a copolymer or blend of PLLA with another polymer or polymers. The polymer or polymers may be polycaprolactone, poly(glycolide), polydioxanone, polytrimethylene carbonate, and poly(4-hydroxybutyrate). Other monomers that can be copolymerized with L-lactide to produce a copolymer are caprolactone, glycolide, dioxanone, and trimethylene carbonate.

It is believed that the mechanisms for reduced angina are due to differences between bioresorbable scaffold-vessel interactions and metal platform stent-vessel interactions. From implantation (t=0) throughout the treatment time until the scaffold completely resorbs, the bioresorbable scaffold induces lower stress on a vessel and lower resultant strain as compared to a metal platform stent.

When a bioresorbable scaffold is implanted, the mechanical properties (such as strength and modulus) and scaffold properties (such as radial strength, radial and axial stiffness) do not change for a period of time, even though the polymer is degrading. After this period, the mechanical and scaffold properties gradually change, for example, the strength, modulus, radial strength, radial stiffness gradually decrease. In order to illustrate this behavior, FIG. 24 depicts a schematic representation of time dependent behavior of a bioabsorbable scaffold after intervention or deployment. The time scale shown is exemplary, the time dependence of scaffold behavior is a qualitative representation. Specifically, FIG. 24 shows the time dependence of the molecular weight of the scaffold material, the radial strength of the scaffold, and the mass loss from the scaffold due to bioresorption of the scaffold material.

The molecular weight of the scaffold decreases with time due to chain scission of the material by hydrolysis. As shown, radial strength does not change for a period of time after implantation in spite of the decrease in molecular weight. However, after this period of time, the radial strength gradually decreases over a period of time. It is believed that polymer fragmentation into segments of low molecular weight polymer due to the scission of amorphous tie chains linking the crystalline regions, results in this subsequent gradual loss of the radial strength. The mass loss is due to assimilation or dissolution of monomers and soluble oligomers resulting from hydrolysis of the polymer. Additionally, the loss of radial strength is followed by a gradual decline of mechanical integrity. The mechanical integrity loss refers to discontinuities in the scaffold struts.

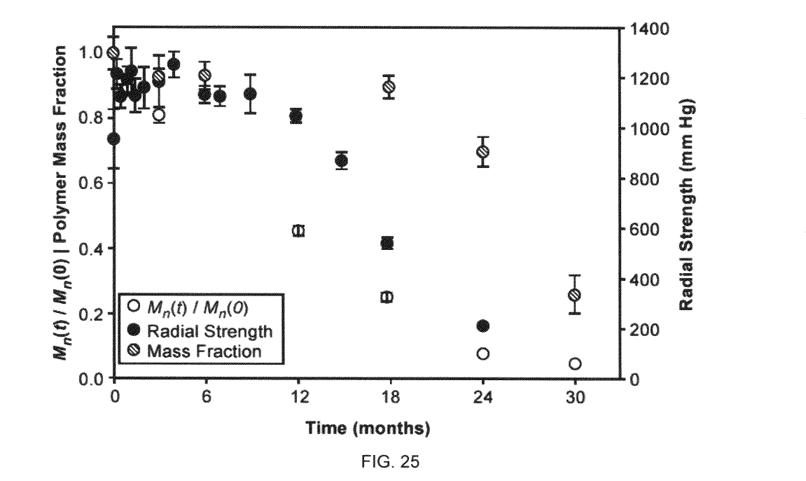

FIG. 25 depicts in vivo and in vitro data for a bioresorbable vascular scaffold made of poly(L-lactide). FIG. 25 shows the normalized molecular weight (Mn(t)/Mn(t=0)), radial strength, and mass fraction, mass (t)/mass(t=0) versus time for a degrading PLLA scaffold. The normalized molecular weight and mass fraction data are in vivo data obtained by implanting the scaffolds in pigs and explanting the samples at time points between t=0 and 30 months. The radial strength data was obtained by soaking scaffolds in saline solution at 37 deg C for the selected time and measuring the radial strength. It is expected that the measured radial strength is not sensitive to an in vitro or in vivo environment.

Therefore, mechanism of action of a bioresorbable scaffold with respect to the reduced stress-strain interaction has two components, a degradation independent component and a degradation dependent component. FIG. 19 illustrates schematically the components of mechanism of action and the time periods that the components are manifested during the treatment time of the bioresorbable scaffold. The time periods shown are exemplary.

Degradation independent component (1) is characterized by low instantaneous stress-strain with the vessel. The low stress-strain interaction is due to the polymer having a lower strength and modulus (stiffness) or lower compliance compared to a metal even in the absence of degradation. As a result, the scaffold exhibits higher compliance when interacting with the vessel, and thus, reduced stress-stress interactions. As indicated, the degradation independent reduced stress-strain interaction persists from t=0 until the properties of the polymer change due to degradation of the polymer. This period may be 30 days PI or longer for example 60 or 90 days PI.

The degradation independent reduced strain component includes greater circumferential conformability, greater axial conformability, and reduced radial compression on the vessel as compared to a metal platform stent. These aspects of the degradation independent stress-strain interactions are discussed in detail herein.

The degradation dependent component (2) is characterized by low stress-strain interaction with the vessel is manifested once the scaffold properties begin to decrease due to degradation of the polymer. The reduction in radial strength and loss of mechanical integrity allow for freedom of radial movement of the stented section of the vessel.

As further illustrated in FIG. 19, unlike a metal platform stent, the freedom of movement allows for a return to vasomotion or pulsatility (3) and positive remodeling or lumen gain (4). The freedom of movement of the vessel results in reduced stress strain interactions.

FIG. 19 also illustrates the time frames of the clinical outcomes and the mechanisms potentially contributing to them. The trend of early SDA-reduction from implantation to about 6 months may be attributed to mechanisms 1 and 2. The significant sustained SDA-reduction from about 6 months to about 2 years and beyond may be attributed to mechanisms 3 and 4. The reduction in TLR and MACE from about 1 to about 2 years and beyond may be attributed to mechanism 2, 3, and 4.

Like traditional (non-degradable polymeric or metallic-backbone DES) tissue engineering (TE), a BVS implant elicits physiological benefit via a) physicochemical induction and b) tissue conduction from around the strut milieu and covering the strut area. However, a BVS provides a distinctive TE cue to the cellular environment as following: a) physicochemical induction includes degradation product due to chain scission, such as smaller than initial molecular weight (e.g., polylactic acid, polylactic acid oligomers, and lactic acid); b) tissue conduction is described as surface dependent at t=0 but also degradation dependent, hence there is time-dependent, evolution of surface texture; and c) mechanical conditioning described as time dependent stress profile to the vessel. This includes an initial stress episode to the vessel followed by degradation dependent, hence time-dependent, reduction in stress to the vessel.

Mechanical conditioning (a) is described as time-dependent reduction in load-bearing capacity derived from controlled and gradual degradation of polymer. Mechanical conditioning and conductive absorbable polymeric surface (b) combines to elicit a composite TE response, resulting in restoration of vascular function and flow close to native state. Mechanical conditioning and tissue conduction are TE phenomena that distinguish BVS implants from non-degradable implants.

The radial stress compression stress (σ_{c}) on the vessel due to struts during the time frame depicted in FIG. 19 is σ_{c}cΔR, where c=proportionality constant and ΔR=change in local radius in contact with strut due to compression . The circumferential stress (σcirc) on the vessel due to struts during the time frame depicted in FIG. 19 is σcirc ≈σ_{c}fD/t, where f=area fraction of vessel area in contact with strut (strut footprint area over total vessel area), D=vessel diameter, and t=vessel thickness.

There are several dimensions to the physiological effect arising from the distinguishing functionality of the BVS. The mechanical conditioning and tissue conduction creates and sustains vascular patency, and consequently perfusion. It confers vascular safety by accelerated and functionally competent endothelium.

The time-dependent load bearing property, as compared to a metallic DES, results in: i) reduction in multi-modal stresses to vessel at all times, ii) freedom of vessel wall motion (vasomotion), iii) positive vessel remodeling without late acquired malapposition, and iv) plaque morphological alteration (plaque regression).

The physiological phenomena of the mechanical conditioning and conductive absorbable polymeric surface of the BVS result in unique flow enhancement due to reduction in flow impedance at implant site and at distal circulation. This clinically manifests in reduction of site diagnosed angina (SDA).

As a result, mechanical conditioning and conductive absorbable polymeric surface combines to elicit a composite TE response, resulting in restoration of vascular function and flow close to native state.

In the EXTEND trial, at one year significant changes in vasoconstriction and vasodilatation were observed during the same test Onuma Y, et al., Circulation 2011; 123:779-97. With respect to lumen gain, between 1 and 3 years, the OCT assessment documented an enlargement of the scaffold area (1.13 mm2) in parallel with an increase in neointima between and on top of the struts (0.94 mm2). The net result is on average an unchanged mean lumen area.

The hypothesized mechanism for inhibition of angina is reduced stress-strain interactions arising individually from the components and a composite of the components. FIG. 19 further illustrates the clinical outcomes from the two components of the reduced stress-strain interactions. During the degradation independent period a trend emerges of early reduction in angina as manifested in the clinical trial. A difference between SDA between ABSORB and the metal platform stents emerged around 30 days PI. The difference was reduced SDA as compared to the metal platform stents. The difference continued to grow during the degradation dependent period.

Referring again to FIG. 19, the clinical results show significant sustained reduction in SDA as compared to the metal platform stents. In addition, although not shown, there was a reduction in target region revascularization (TLR) and major adverse cardiac event (MACE).

Table 4 summarizes three distinct phenomena and hypotheses of mechanisms by which phenomena contribute individually and in combination to angina reduction. Phenomena 1 (P1) relates to increased acute (e.g., 0-30d) radial and axial compliance observed for Absorb vs XV at t=0, hence reduced stress-strain on vessel at t=0. Phenomena 2 (P2) relates to increased vasomotion observed starting 6 months after Absorb implantation compared to post-PCI. Phenomena 3 (P3) relates to vessel benign positive remodeling observed between 6 and 12 months' time point and 6 months of ABSORB implantation compared to post-PCI.

TABLE 4
Summary of phenomena and hypotheses contributing to reduced angina for a
bioresorbable polymer scaffold.

| Hypothesis | Phenomena 1 (P1) | Phenomena 2 (P2) | Phenomena 3 (P3) |
|---|---|---|---|
| I. P2 + P3 additively enhances flow rate | Increased acute (0-30 d) | Increased vasomotion | Vessel benign |
| between about 6-12 mos; reducing SDA. | radial and axial | observed starting 6 | positive remodeling |
| II. P2 enhances flow rate starting 6 mos | compliance observed for | months of Absorb | observed between 6 |
| III. P1 provides optimal stress-strain | Absorb vs. XV at t = 0, | implantation compared | and 12 month time |
| equilibration during vessel scaffolding at | Hence less stress-strain | to post-PCI. | point. 6 months of |
| t = 0 | on vessel at t = 0. | | Absorb implantation |
| IV. P1 and P2 promotes, additively or | | | compared to post- |
| synergistically, functional neo | | | PCI. |
| media/endothelium starting 6 months, | | | |
| resulting in benign positive remodeling | | | |
| V. Absorb load bearing property as a f(t) | | | |
| allows for scaffold to conform to vessel | | | |
| during benign positive remodeling without | | | |
| malapposition. | | | |
| VI. All of the above (I-V) follow | | | |
| superposition principle and additively or | | | |
| synergistically contribute to reduction of | | | |
| SDA at 1 yr and potentially reduction of | | | |
| TLR and MACE at longer terms, >12 | | | |
| months | | | |
| VII. P1 contributes to acute safety | | | |

Hypothesis I is that P2+P3 additively enhance blood flow rate between about 6 to 12 mos; reducing SDA. The greater freedom to radial fluctuation and positive vessel remodeling increases blood flow rate in ABSORB vessels as compared with reference flow rate for a stented vessel.

Hypothesis II is that P2 enhances flow rate starting about 6 months since the pulsatility allows for increased flow rate through the vessel even in the absence of positive remodeling.

Hypothesis III is that P1 provides optimal stress-strain equilibration during vessel scaffolding at t=0 and extending through a period in which scaffold properties are independent of degradation. The bioresorbable scaffold reduces stress-strain interactions while maintaining patency. This is in contrast to a metal platform stent which has lower circumferential and axial conformability than a bioresorbable polymer scaffold. The optimal stress-interactions with the vessel contribute to reduction in angina and non-ischemic chest pain. The mechanisms for reduction are described herein.

Hypothesis IV is that P1 and P2 promotes, additively, or synergistically, functional neo-media/endothelium starting about 6 months, resulting in benign positive remodeling. The 6 month time to endothelialization was demonstrated in a preclinical model and in clinical studies. Functional neo-media/endothelium is sufficiently strong to maintain an increased diameter or patency and also undergo vasomotion. The neo-media/endothelium is allowed to strengthen during the period scaffold properties are independent of degradation and the strengthened endothelium and adjust to normal pulsatility.

Hypothesis V is the that bioresorbable load bearing property as a function of time, f(t), allows for scaffold to conform to vessel during benign positive remodeling without malapposition.

Hypothesis VI is that all of the above (I-V) follow a principle and additively or synergistically contribute to reduction of SDA at 1 yr and potentially reduction of TLR and MACE at longer terms, e.g., greater than 12 months

Hypothesis VII is that P1 contributes to acute safety.

Expressed in mathematical terms, angina reduction can be expressed as:

Angina Reduction=f₁(P2)+f₂(P3) (1)

f₂(P3)=g₁(P1)+g₂(P2) (2)

f₁(P2)=h₁(P1) (3)

where f₁, f₂, g₁, g₂, and h₁ are undetermined functions of the phenomena, P1, P2, and P3.

Benign Positive Remodeling (BPR) refers to an expansion in the external elastic lamina (EEL) without aneurysmal dilatation and includes the following aspects:
(1) Increase in lumen area;
(2) Maintenance of medial integrity without excessive compression or injury;
(3) Optimal leukocytic involvement/modulation of vascular response, allowing for timely (3-6 months) re-endothelialization and restoration of endothelial and smooth muscle cell (SMC) function.
   (a) Leukocytes (e.g. M2 macrophages) are integral parts of tissue remodeling and are therefore required for BPR to occur;
   (b) Leukocytes are an integral part of aliphatic polyester (e.g., PLLA-based polymer) resorption, as based on (a) above with connective tissue integration into regions previously occupied polymer;
   (c) Non-optimal or adverse leukocytic involvement may result in excess neointimal proliferation (lumen loss despite EEL area expansion), excess injury to the arterial wall/media, and/or pathological positive remodeling (aneurysmal dilatation, malapposition).

With regard to Hypothesis IV in which P I contributes to BPR and P2, the increased radial and axial compliance at t=0, hence lower stress and strain to vessel, provided by a bioresorbable stent such as Absorb relative to metallic platforms has several consequences described below.
1. Promotes the more rapid equilibration of vessel strain close to native state, thus promotes more normal endothelial cell and SMC function (required for P2/vasomotion),
2. Allows for a reduction of medial compression/injury to the arterial wall, thus reducing post-implant/injury-related to inflammation (leukocyte infiltration), this leads to optimal leucocyte modulation. Leukocyte modulation is inherent part of all interventional procedures, and a reduction of post-implant injury leads to an optimization of leukocyte modulation (as opposed to non-optimal or adverse, 3c above).
   a) Optimal leukocytic modulation contributes to BPR.
   b) A reduction in inflammation effectively promotes re-endothelialization and phenotypic maturation of SMCs from a proliferative (immature) to a contractile (mature) phenotype (inflammation is associated with increased neointimal proliferation, thus signaling SMCs to remain in a proliferative phenotype).

Aneurysmal dilatation is defined as an increase in EEL and/or lumen area of greater than 50% of the implanted region as compared to the respective areas within the proximal or distal reference vessel.

The present invention includes basing treatment and treatment recommendations for CAD with a bioresorbable stent such as a bioresorbable polymer stent on reduced stress with such polymer stents which will result in reduction of prevention of angina as a result of such reduced stress as compared to metallic platform stents.

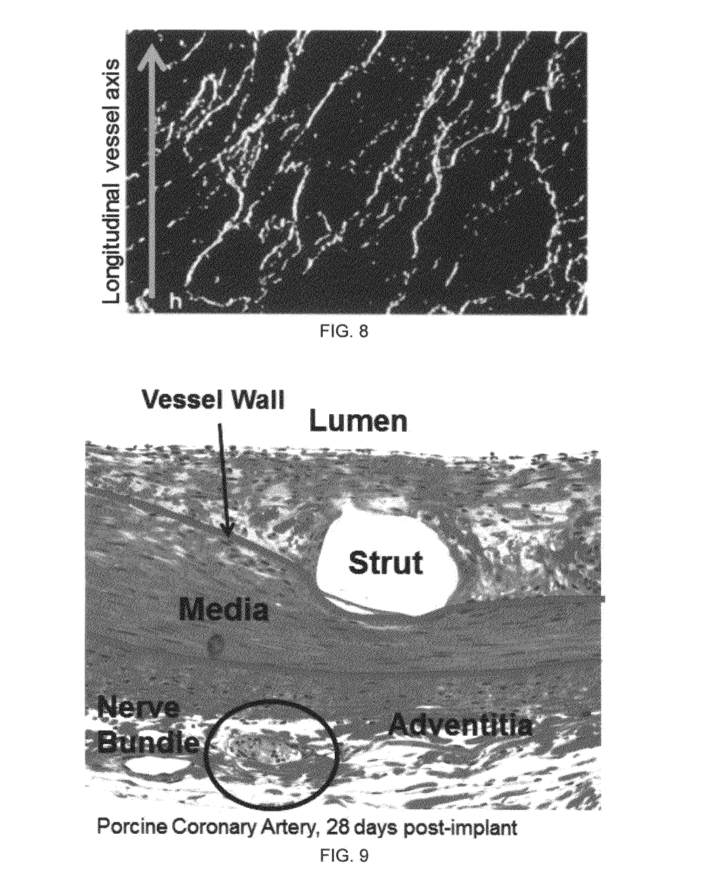

FIG. 8 depicts a confocal scanning laser microscopy image of a longitudinal section of a porcine coronary artery stained with a non-specific marker for neural cells 28 days post intervention with a stent. Buwalda et al., J Neuroscience Methods 73: 129-134, 1997. FIG. 9 depicts a lumenal view of the porcine coronary artery of FIG. 8, 28 days post implant. In FIG. 9, a strut is shown in contact with the lumen wall making a small depression into the media which is adjacent to the adventitia. Nerve bundle or nerve plexus, which is a network of intersecting nerves, is shown adjacent to the adventitia, which can be stimulated by mechanical stresses and induce angina.

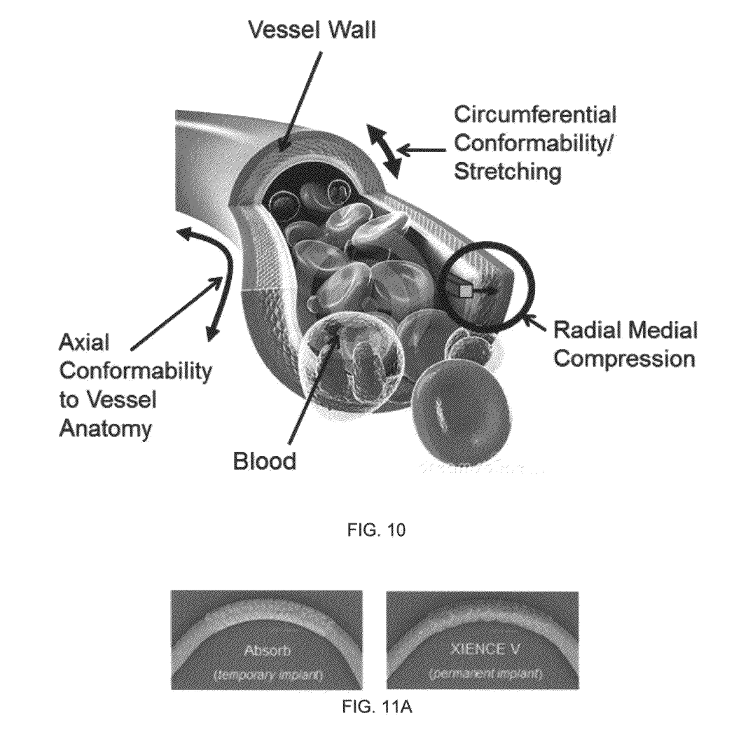

FIGS. 10-18 illustrate aspects of P1, degradation independent reduced stress-strain interactions. As described herein, the bioresorbable stent provides for optimal stress-strain interactions at t=0 with a vessel as compared to a metal platform stent. The bioresorbable stent provides better axial and circumferential conformability to a vessel as compared to a metal platform stent which results in reduced stress-strain interactions with the vessel, where stress-strain refers to that of the vessel. Non-optimal stress strain interactions are hypothesized to contribute to angina, as described above. Aspects of stenotic vessel that may contribute to non-optimal stress-strain interactions may be indicators or predictors of angina. Non-optimal stress strain interactions are further hypothesized to contribute to non-ischemic chest pain. The increased axial conformability or compliance and circumferential (or radial) conformability or compliance are believed of the bioresorbable stent are believed to reduce or prevent non-ischemic chest pain by mechanisms as described herein.

One aspect of the present invention is that stresses imposed by the stent on the vessel stimulate the nerve bundle and induce chest pain. This hypothesis is based on arterial mechanical effects of PCI with a stent.

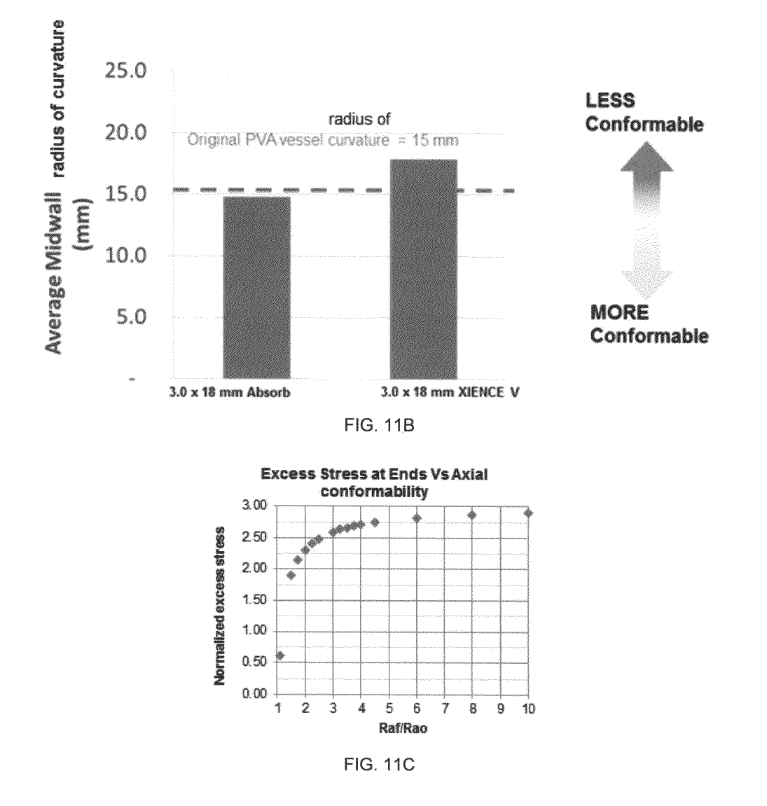

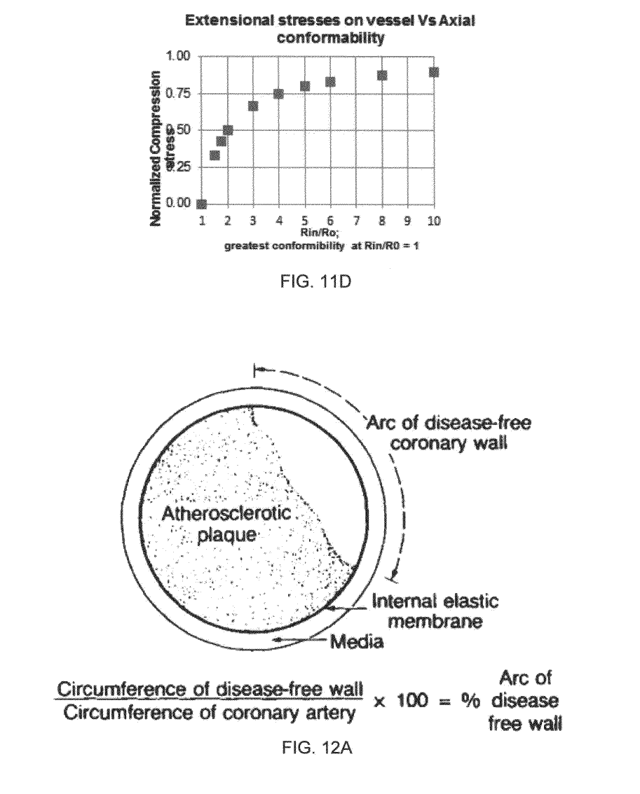

FIGS. 10 and 11A-11C illustrate higher axial conformability of the bioresorbable scaffold as compared to a metal platform stent.

FIG. 10 depicts a cut-away section of a blood vessel illustrating arterial mechanical effects of PCI with a stent. Axial conformability to vessel anatomy is the ability of a vessel to maintain natural axial profile which can be shown by the degree to which the stent alters an axial profile, specifically, curvature. When a stent is implanted, the natural curvature of the vessel can be altered by force forces imposed by the stent. For example, an implanted stent which has a straight axial profile may decrease the curvature of a vessel segment. The natural profile may be that which provides the least stress to the vessel wall. The greater the change in the curvature, the greater the increase stress which results in greater stimulation of nerve bundles adjacent to the vessel wall.

The change in curvature of a vessel post implant from a pre-implant curvature of the vessel for a bioresorbable polymer implant may be less than 15%, less than 10%, less 5%, or less than 1%.

It is hypothesized by the present invention that pre-implant vessel curvature, which is the inverse of radius of curvature, is an indicator of angina, for example, a pre-implant curvature greater than 0.05 cm⁻, 0.1 cm⁻¹, 0.2 cm⁻¹, 0.3 cm⁻¹, 0.4 cm⁻¹ or 0.5 cm⁻¹. The prevention or degree of reduction of stress may depend on characteristics of the vessel. For axial conformability, the greater the pre-implant vessel curvature, the greater may be the reduction in stress. A straight vessel or one with very low curvature may have no or very low axial stresses from a straight stent implantation.

Circumferential Conformability is the ability of a stent to conform or adapt to a natural vessel circumferential shape or profile which may minimize stress on the vessel wall. The circumferential profile of the stent is circular, however, the natural profile of the vessel may depart from a circular shape, for example, an oval or flattened circle. The greater the change in the natural profile, the greater the increase stress which results in greater stimulation of nerve bundles adjacent to the vessel wall.

A bioresorbable polymer stent may also have better circumferential conformability that a metal platform stent arising from a difference it manufacturing. Specifically, a bioaborbable polymer stent may be formed by laser cutting a tube at a diameter close to the deployed diameter in a vessel (e.g., Dcut/Ddep=0.9 to 1.3), an exemplary Dcut being 3.5 mm. A metallic stent may be laser cut from a tube much smaller than a deployed diameter (e.g., Dcut/Ddep=0.5 to 0.7), an exemplary Dcut being 1.9 mm. For a 1.9 mm laser-cut metallic stent, expanding a 6 hinge design beyond 1.9 mm means the structure must stretch into a hexagonal deployment shape when coming into contact with a smooth 3.0 mm vessel, leading to high stress at each of the 6 hinge points around the circumference. Expanding a polymer stent with Dcut of 3.5 mm will better match the circumferential curvature of the vascular wall, which potentially leads to lower induced stresses even with a thicker strut.

It is hypothesized in the present invention that plaque eccentricity may be an indicator of angina due to circumferential stresses upon implantation. The eccentricity index may be calculated by the formula: (Max wall thickness−Min wall thickness)/Max wall thickness. A lesion may be defined as eccentric if the index was >0.5 and as concentric if ≦0.5.

Radial Medial Compression refers to the local compression of the media by the struts. The greater the change in the compression, the greater the increase stress which results in greater stimulation of nerve bundles adjacent to the vessel wall.

It is believed that for some patients ABSORB induces lower forces on the vessel as compared with metallic DES. As a result, ABSORB induces lower stimulation of the peri-adventitial nerve tissue than the metallic DES stents. The lower stimulation may result in reduction or prevention of stent-induced angina in some patients.

ABSORB shows better axial conformability to vessel anatomy than a DES metallic stent. ABSORB deploys with minimal changes to vessel longitudinal anatomy (curvature), resulting in lower axial stresses on the implanted vessel segment.

With regard to circumferential conformability, stenting a vessel necessarily involves expansion and providing patency to stenotic section of a vessel. However, a bioabsorbable scaffold such as ABSORB balances patency vs. strain and ABSORB provides optimal acute gain sufficient for re-establishing flow, but lower than that observed in extreme metal DES expansions, resulting in lower circumferential strain and injury.

With regard to medial compression, the struts of ABSORB have a larger footprint as compared to DES metallic stents. The larger footprint reduces localized (under-strut) compression of the media. The larger footprint of ABSORB is due to a strut width of about 165 microns. The footprint or strut width for the DES is much smaller, e.g., 70 to 100 microns or 70 to 120 microns. Taxus Liberte has a strut width of 76 microns with a 20 micron coating. Taxus Express has a strut width of 91 microns with a 22 micron coating. XV has a strut width of 94 microns with an ablumenal coating thickness of 7.8 microns. As a result of the larger footprint; the radial compression of the struts is spread over a larger vessel wall area which results in reduced radially inward compression and thus stress.

Additionally, the larger footprint of the larger struts is shown by the scaffold artery ratio (SAR) for the stent which for the ABSORB scaffold of the clinical studies is approximately 24%, as compared to the SAR for XV (3mm×18 mm) which is 13.7%. The SAR of a polymer scaffold may be 1.5 to 2.5 or 1.8 to 2.2 times a metal platform scaffold.

FIGS. 11A-B compares the axial conformability to vessel anatomy of ABSORB and XV. An ABSORB scaffold and an XV stent were deployed in a PVA vessel having an original midwall radius of curvature of 15 mm. FIG. 11A depicts ABSORB and XV deployed in the curved PVA vessels.

FIG. 11 B depicts the average midwall radius of curvature of the deployed ABSORB scaffold and XV stent. An increase in radius of curvature (a decrease in curvature) corresponds to less conformability to the vessel while a decrease in radius of curvature (increase in curvature) corresponds to greater conformability. As shown in FIG. 11B, the XV stent has increased the radius of curvature by about 15%, or equivalently, decreased the curvature of the PVA vessel. ABSORB, on the other hand, has maintained the native radius of curvature within 5%. Thus, the ABSORB is shown to be more axially conformable to a vessel, while the DES metal stent results in higher stressed state due to an increase in radius of curvature or reduction in vessel curvature away from a natural state.

As indicated, it is desirable for a bioabsorbable scaffold to balance patency vs. strain and provide optimal acute gain sufficient for re-establishing flow, but lower than that that would result in lower circumferential strain and injury. In general, scaffold may be designed so that it is not too strong, but strong enough to provide an optimal post-PCI lumen/vessel size. One way to set a design bound is through Glagov's compensatory remodeling observations, Glagov et al., N Engl J Med. 1987 May 28; 316(22):1371-5. The observations indicate that native vessels lose their ability to positively remodel when accumulation of plaque encompasses 40% of the vessel area. In order to encourage a blocked vessel (e.g, 75% blocked) to remodel again, it is likely that an optimal scaffold will have to return this blockage severity back to below 40%. A scaffold may be designed so that it has the strength and stiffness sufficient to reduce blockage to below 40% without inducing high stress in the vessel that would induce chest pain. Such an optimally strong scaffold may result in a smaller lumen, but is sufficient to encourage positive remodeling.

In Gomez-Lara, a study compares 102 patients who received an MPS (Xience V) in the SPIRIT FIRST and II trials with 89 patients treated with cohort B ABSORB. Gomez-Lara, J. et al., JACC CI 3,11 (1190-8), 2010. All patients were treated with a single 3×18 mm device. Curvature and angulation were measured with dedicated software by angiography.

Both BVS and MPS had significant curvature and angulation upon implantation, however, BVS had better conformability than MPS. BVS tended to restore the coronary configuration to that seen before implantation. The coronary geometry remained similar to that seen just after implantation with MPS.

Table 3 summarizes some of the results. Both the MPS and BVS groups had significant changes in relative region curvature (MPS vs. BVS: 28.7% vs. 7.5%) and angulation (MPS vs. BVS: 25.4% vs. 13.4%) after deployment. The unadjusted comparisons between the 2 groups showed for BVS a nonsignificant trend for less change in region curvature after deployment (MPS vs. BVS: 0.085 cm(-1) vs. 0.056 cm(-1), p=0.06) and a significantly lower modification of angulation (MPS vs. BVS 6.4° vs. 4.3°, p=0.03). By multivariate regression analysis, the independent predictors of changes in curvature and angulation were the pre-treatment region curvature, the pre-treatment region angulation, and the used device.

TABLE 3
Summary of changes in curvature for BVS and metallic stents.

| | | | | | Relative | | | |
|---|---|---|---|---|---|---|---|---|
| | | | | | Changes | | | |
| | | | | | Pre vs. | | | |
| Variable | Device | Pre-Treatment | Balloon | Post-Treatment | Post (%) | p Value | p Value^{†} | p Value [symbol] |
| Curvature (cm⁻¹) | BVS | 0.292 (0.179-0.576) | 0.135 (0.073-0.276) | 0.270 (0.114-0.429) | 7.5 | <0.01 | <0.01 | 0.06 |
| | MPS | 0.324 (0.159-0.571) | 0.117 (0.051-0.272) | 0.231 (0.123-0.400) | 28.7 | <0.01 | <0.01 | |
| Angulation (*) | BVS | 29.6 (15.82-55.4) | 6.8 (1.8-14.8) | 25.6 (12.6-43.1) | 13.4 | <0.01 | <0.01 | 0.03 |
| | MPS | 38.1 (21.1-60.8) | 8.2 (2.8-15.9) | 28.5 (14.5-45.7) | 25.4 | <0.01 | <0.01 | |
| Cyclic changes in | BVS | 0.097 (0.035-0.190) | — | 0.072 (0.034-0.155) | 25.8 | 0.82 | — | 0.01 |
| Curvature (cm⁻¹) | MPS | 0.091 (0.051-0.173) | — | 0.056 (0.023-0.092) | 38.5 | <0.01 | — | |
| Cyclic changes in | BVS | 4.7 (1.9-11.7) | — | 4.6 (2.2-8.7) | 1.7 | <0.01 | — | 0.04 |
| Angulation (*) | MPS | 6.4 (2.7-11.4) | — | 3.8 (1.7-6.3) | 41.0 | <0.01 | — | |

(*) Comparisons are made within groups comparing pre- and post-treatment value;
^{†}comparisons are made within groups comparing pre, balloon and post-treatment values; [symbol]
Comparisons are made between groups comparing the mean changes pre-post of each group.

ABSORB axial conformability is greater than that of XV. The present invention indicates that greater axial conformability produces less stress on vessel:

(1) Vessel straightening produces compressive and extensional stresses on the implanted vessel segment;

$${δ}_{b} = \frac{Ey}{L} ; \frac{{δ}_{b}}{E} = ( 1 - \frac{ri}{ro} ) ,$$

where δ_{b}=extensional stress, compressive stress, rᵢ=inner radius, rₒ=outer radius. Radii are of curved vessel segment that straightens when implanted; E=vessel Young's modulus; y=deformation due to decrease in curvature; L=length of stent after deployment. Deformation “γ”=vessel straightening, i.e., change from a greater curved contour to a straight contour, is higher for XV vs ABSORB.

(2) Vessel straightening produces excess stresses at the proximal and distal ends of the implanted vessel segment;

$${δ}_{a} ≅ {k ( \frac{1}{{r}_{ao}} - \frac{1}{{r}_{af}} )}^{n} ; {r}_{af} > {r}_{a 0} ;$$

where k is a constant. For completely straightened vessel r_{af}=∞; δₐ=excess extensional and compressive stress at the ends of the implant.

FIG. 11C-D show a relationship between axial conformability and stress obtained from the analytical model described above. FIG. 11C depicts the normalized excess stress vs. initial radius of curvature at the end of stented segment. FIG. 11D depicts the normalized extensional stress vs. initial radius of curvature along the axis of stented segment.

BVS vessel at 6 months via IVUS indicates more conformability axially and less circular radially vs. XV. Assuming axial conformability does not change between 0 and 6 months, this woul d result in less axial strain both acutely (t=0) and subacutely (t=0-30 days).

The data indicates greater axial conformability of Absorb produces less vessel straightening and correspondingly: less extensional stresses on the implanted vessel segment and less excess stresses at the proximal and distal ends of the implanted vessel segment. Clinical Data shows Absorb more conformal axially vs. XV, at post-procedure.

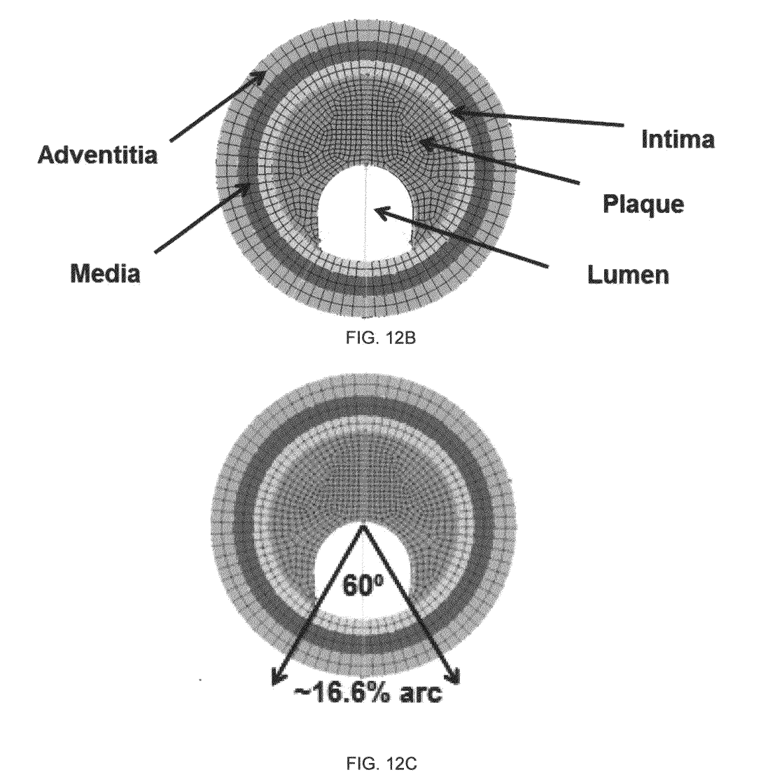

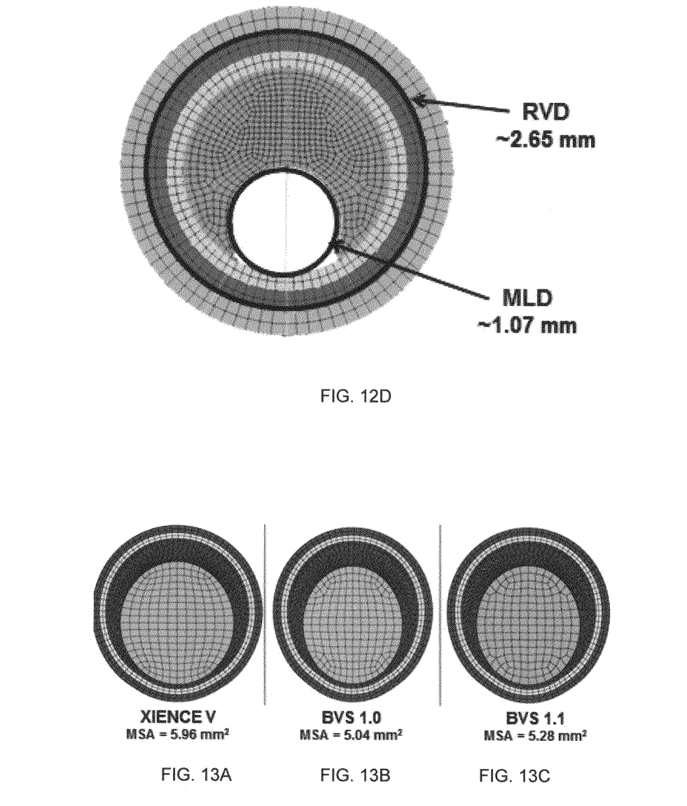

The circumferential conformability/stretching was studied by creating a relevant eccentric lesion finite element model. FIG. 12A depicts a diagram showing the definition of disease-free segment of an eccentric lesion and the calculation of the percent of disease-free circumference. (Waller et al., Clin. Cardiol. 12, 14-20 (1989)) Eccentric lesions impart uneven loading on a deployed scaffold that are thought to produce scaffold eccentricity. Waller et al. described 365 of 500 lesions as eccentric which were 73% of the segments measured. Average lesion geometry had 16.6% arc (60°)disease-free wall.

FIG. 12B depicts the finite element model of a vessel that includes the adventitia, media, intima, and plaque. Artery and plaque material properties were assigned according to work by Zahedmanesh and Holzapfel. Zahedmanesh H, et al. Med Bio Engr & Comp. 2009; 7:385-393; Holzapfel G , et al., Am J of Phys. Heart and circulatory physiology. 2005; 289:H2048-2058. Thicknesses for arterial layers were defined per Holzapfel (Intima+Media+Adventitia total ˜0.55 mm). FIG. 12C depicts the finite element model of FIG. 12B with the disease-free arc labeled.

Lumen and vessel border were sized to be equal to pre-procedural mean lumen diameter (MLD) and reference vessel diameter (RVD), respectively, from Brugaletta's eccentricity study (Brugaletta S., Catheterization and cardiovascular interventions : official journal of the Society for Cardiac Angiography & Interventions. 2012; 79:219-22). FIG. 12D depicts the finite element model of FIG. 12B with the MLD and RVD labeled.

Deployment was simulated for BVS 1.0, BVS 1.1, and XV. The eccentricity index at minimum scaffold area (MSA) for each was taken from Brugaletta et al.: BVS 1.0 EI=0.83; BVS 1.1 EI=0.85; and XV EI=0.90. The major axis radius (a) and minor axis radius (b) of an oval or ellipse for each is:

BVS 1.0 a=1.39 mm, b=1.15 mm

BVS 1.1 a=1.41 mm, b=1.20 mm

XV a=1.45 mm, b=1.31 mm.

The XV is more circular than the BVS. BVS 1.0 is more ovalized than BVS 1.1.

Based on these two parameters (MSA and EI), idealized deployment ovals were constructed for BVS 1.0, BVS 1.1, and XIENCE V. Deployment in an eccentric lesion was simulated to study effect of BVS and XIENCE V deployed geometries on vessel stress levels.

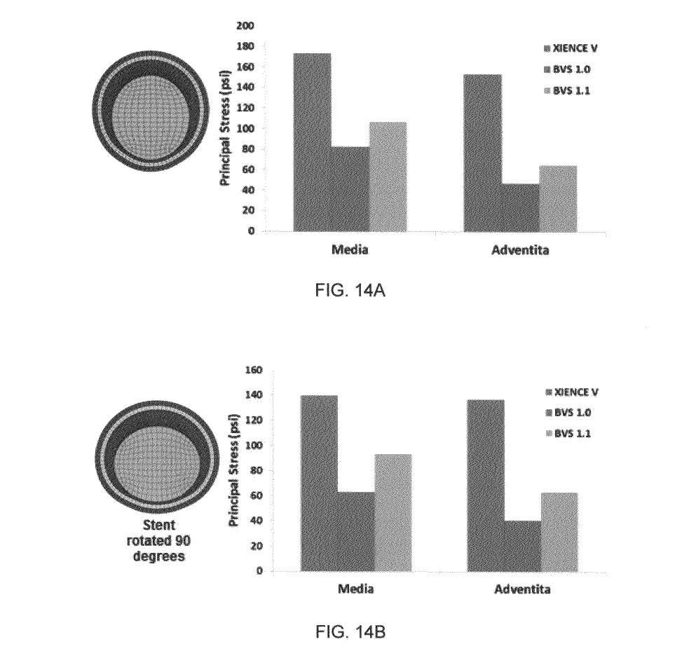

Deployment was simulated by imposing deployment ovals from Brugaletta et al. with appropriate dimension for major and minor axes, “a” and “b”, respectively, calibrated to eccentricity index (El) and MSA for XV and BVS. FIGS. 13A-C and 14A-B depict the results of simulated deployment of XV and BVS based on the model of FIG. 12B. FIGS. 13A-C depict the simulated model for XV, BVS 1.0, and BVS 1.1, respectively, post-deployment. In each case, the vessel models show a degree of eccentricity (i.e., deviation from circular cross-section). The BVS each appear to have a higher degree of eccentricity.

Stresses in the arterial layers were compared in two deployment orientations, 0° and 90°, with respect to the eccentric lesion. FIG. 14A compares the circumferential stress of XV, BVS 1.0, and BVS 1.1 in both the media and adventitia at an orientation of 0°. FIG. 14B compares the circumferential stress of XV, BVS 1.0, and BVS 1.1 and ABSORB in both the media and adventitia at an orientation of 90°. The stress is lower for ABSORB in both regions. The stress is about 75% higher in XV over BVS 1.1 in the media and about 130% higher in XV over BVS 1.1 in the adventitia.

The MSA's and eccentricity indices (El) after deployment were lower for the BVS platforms vs. XV. When compared to XV, adventitial stresses were reduced by 69% with BVS 1.0 deployment. When compared to XV, adventitial stresses were reduced by 58% with BVS 1.1 deployment. Similar stress drops for BVS were observed when oval orientation was rotated (70% and 54% stress reductions were calculated).

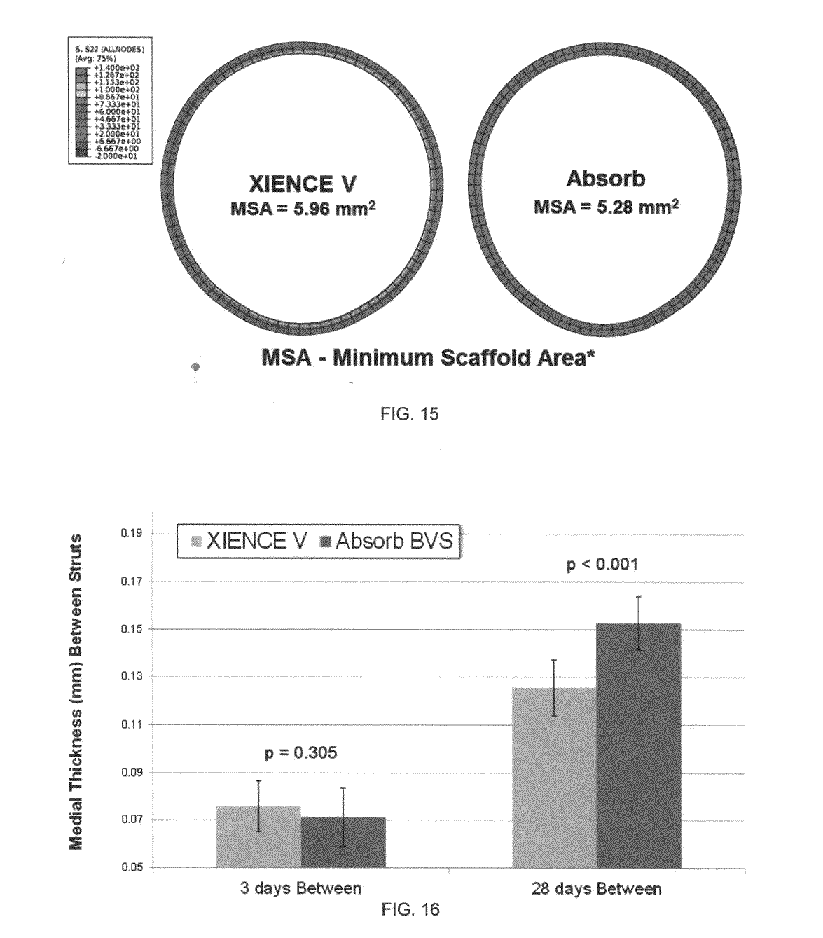

FIG. 15 depicts the media layer of the model the simulated deployed XV stent and ABSORB scaffold with level of stress indicated in the elements. The lower stress in the media layer in ABSORB is shown.

Circumferential Conformability has been studied in a preclinical model and have shown reduced circumferential stress/strain for Absorb vs. Xience V. FIG. 16 depicts the medial thickness between struts for ABSORB and XV in a porcine model at 3 days and 28 days post implantation. The medial thickness for ABSORB and XV is within the error bars at 3 days. However, at 28 days, the ABSORB medial thickness is larger than the XV medial thickness indicating a higher degree of strain in circumferential direction of the vessel.

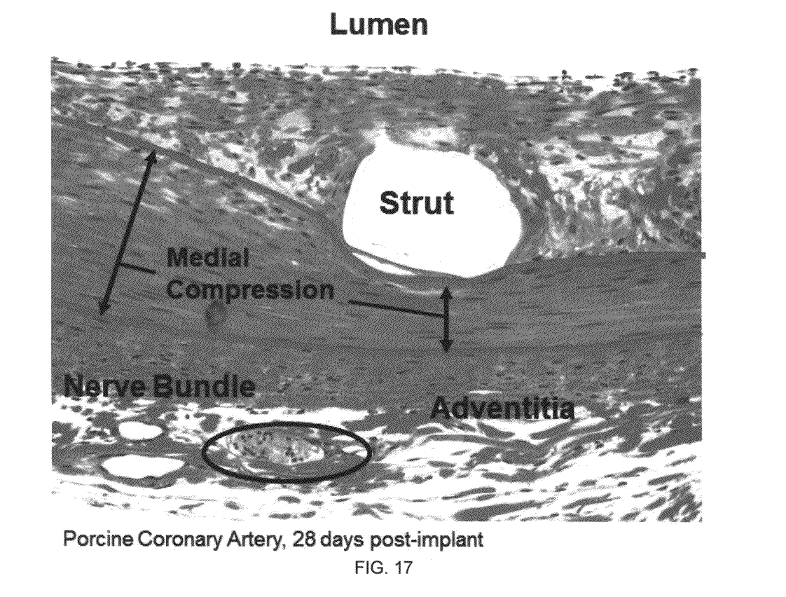

FIG. 17 depicts the lumen view of FIG. 9 of the porcine coronary artery, 28 days post-implant. FIG. 17 shows a depression in the medial layer adjacent to the strut. The depression is compared to the portion of the medial layer between struts.

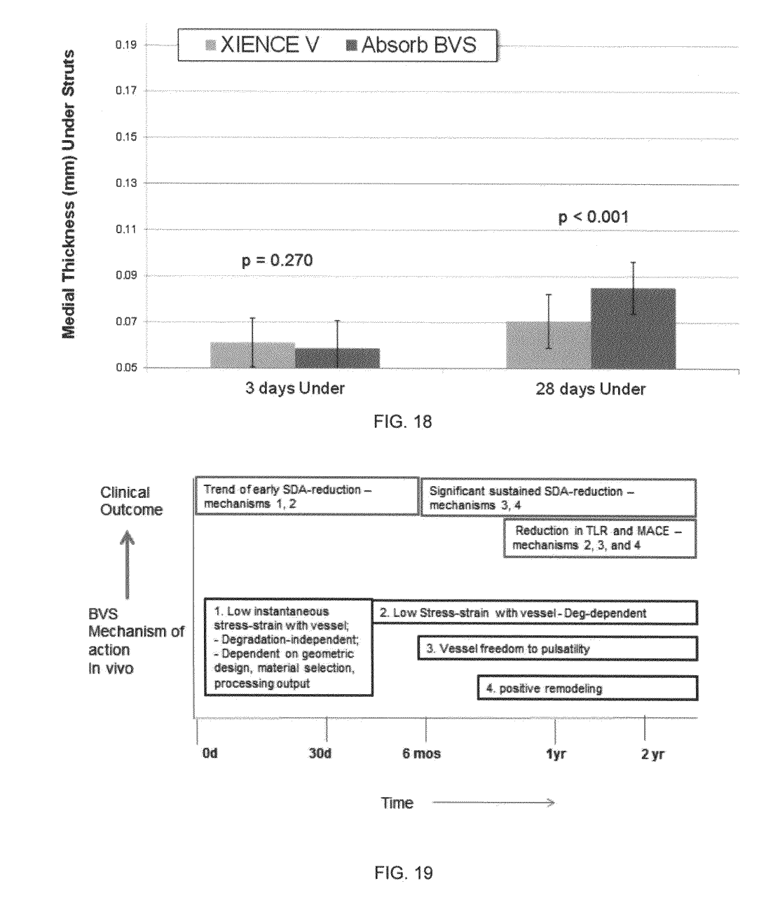

FIG. 18 depicts the medial thickness under struts for ABSORB and XV in a porcine model at 3 days and 28 days post implantation. The medial thickness for ABSORB and XV is within the error bars at 3 days. However, at 28 days, the ABSORB medial thickness is larger than the XV medial thickness indicating a smaller compression of the medial layer as compared to the XV compression.

The incidence of post-procedural chest pain in ABSORB EXTEND appears lower than that observed in metallic DES trials. It is hypothesized that lower stresses placed on the vessel by ABSORB vs. metallic DES results in less stimulation of the peri-adventitial nerves. This hypothesis is supported by the greater observed conformability to the axial anatomy of vessels, confirmed in ABSORB Cohort B patients when compared to SPIRIT I & II Patients. The hypothesis is further supported by lower circumferential stretching/stress upon deployment, deduced from the observed lower acute gain and dimensional outcomes in ABSORB Cohort B patients. The hypothesis is further confirmed by lower radial medial compression, observed in pre-clinical model.

The axial conformability, circumferential conformability, and reduced compression and reduced stress on the vessel by the polymeric scaffold as compared to a metallic stent may be attributed to several factors including scaffold properties such as lower radial stiffness, lower radial strength, lower axial bending stiffness. The scaffold properties are influenced by material properties such as the tensile modulus which is lower for the polymer of the scaffold than a metal. Ranges of tensile modulus of polymers for the scaffold are disclosed herein. The scaffold properties are also influenced by the scaffold pattern which can be modified to reduce radial and axial stiffness. The scaffold properties, material properties of the scaffold, and the scaffold pattern can be modified to provide axial conformability, circumferential conformability, and reduced compression which result on reduced stress on the vessel.

It is hypothesized that any one of the three types of stress or any combination of the three can stimulate the peri-adventitial nerves. Stent induced chest pain may be avoided by a polymer stent when all three mechanisms fail to induce stresses sufficient to stimulate the nerve bundle to cause angina.

A model is provided for increased flow includes metrics for amplitude of fluctuations of the vessel (a) and increment fraction of mean vessel diameter due to remodeling (b). The model assumes laminar flow of Newtonian fluid, sinusoidal radial fluctuation and scaffold stiffness decreases amplitude of fluctuations, “a”. A radially compliant scaffold allows for larger amplitude oscillations “a” and hence increases the flow or decreases the impedance. The relevant model for the flow rate in a vessel is as follows:

$${Q}_{ref} = \frac{π ∇ {PR}_{m 0}^{{4}^{□}}}{8 μL} ; {Q}_{av} = \frac{{Q}_{ref}}{T} {\int }_{0}^{T} {( 1 + a Sin wt )}^{4} d t ;$$

where Q_{ref} is the flow rate in a stented vessel of the same Rₘ₀ and Qₐᵥ is the average flow rate over a time period (T) of one cycle of vessel pulsatile radial fluctuation, ∇P/L=pressure gradient, Rₘ₀=radius of the stented segment post-stenting, μ is the viscosity, ω=2πf with f=frequency of vessel pulsatile radial fluctuation. For a <<1, Qav≈Qref(1+3a+b²) so the overall flow rate for BVS vessel segment is Qav≈Q_{ref}(1+b)⁴ (1+3a²).

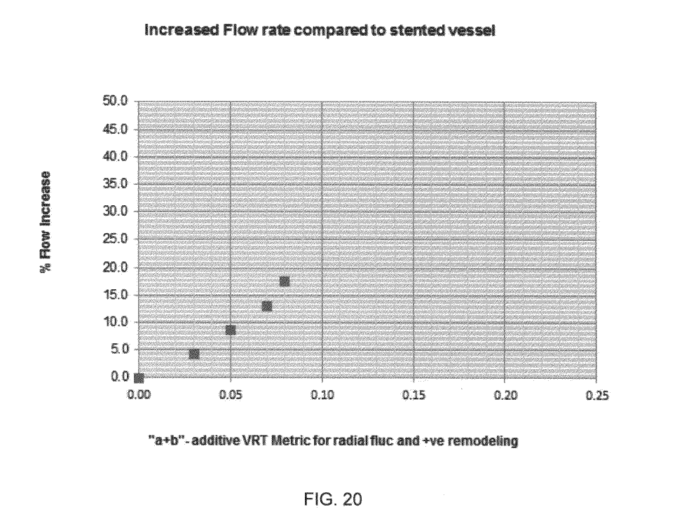

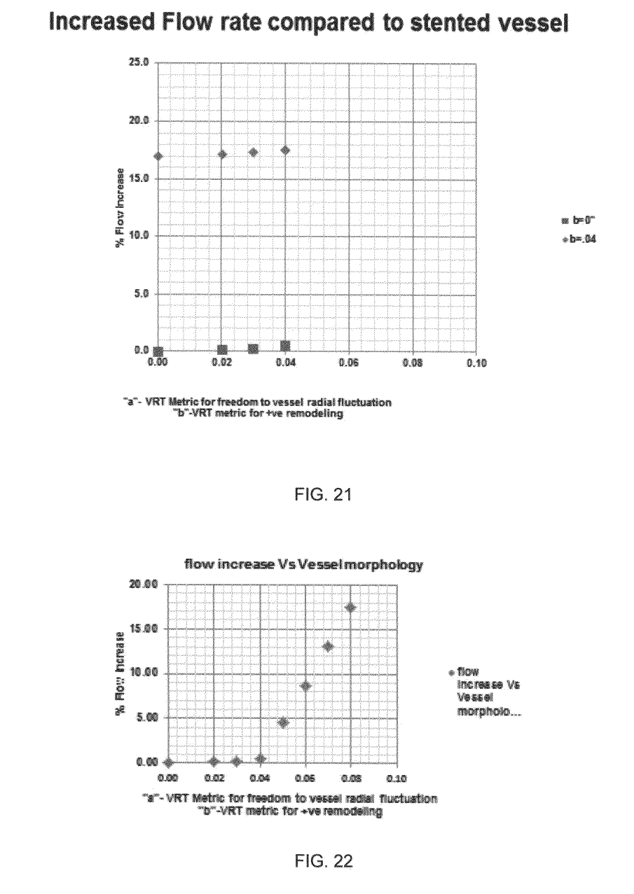

FIG. 20 depicts the percent increased flow rate compared to the stented vessel vs. “a+b” additive vascular restorative theory (VRT) metric for radial fluctuations and positive remodeling. FIG. 21 depicts the percent increased flow rate compared to the stented vessel vs. “a” for two values of “b,” larger flow rate increase for b=0.04, smaller flow rate increase for b=0.

FIG. 22 depicts the percent increased flow rate compared to the stented vessel vs. “a+b” additive vascular restorative theory (VRT) metric for radial fluctuations and positive remodeling based on the data in Table 5.

TABLE 5
VRT metrics for radial fluctuations and positive remodeling.

| Radial | positive | | |
|---|---|---|---|
| fluctuation | remodel % | % Increase in Flow | a + b = VRT metric as |
| a | b | from stented reference | f(t) |
| 0.00 | 0.00 | 0.00 | 0.00 |
| 0.02 | 0.00 | 0.12 | 0.02 |
| 0.03 | 0.00 | 0.27 | 0.03 |
| 0.04 | 0.00 | 0.48 | 0.04 |
| 0.04 | 0.01 | 4.56 | 0.05 |
| 0.04 | 0.02 | 8.76 | 0.06 |
| 0.04 | 0.03 | 13.09 | 0.07 |
| 0.04 | 0.04 | 17.55 | 0.08 |

One aspect is the use of a polymer, in particular a bioresorbable polymer, for the scaffold. A polymer scaffold may be less traumatic to a vasculature. Polymers are softer, less stiff or have a lower modulus than metals. Thus, the presence of a softer, more flexible implant may be less traumatic to a soft, flexible vessel segment than a metal implant. For example, aliphatic bioresorbable polymers have tensile moduli generally less than 7 GPa and in the range of 2 to 7 GPa (US2009/0182415). Poly(L-lactide) has a tensile modulus of about 3 GPa.

Metals used to make a stent and their approximate moduli include stainless steel 316L (143 GPa), tantalum (186 GPa), Nitinol or nickel-titanium alloy (83 Gpa), and cobalt chromium alloys (243 Gpa). These moduli are significantly higher than aliphatic polymers. The strengths of these metals are also significantly higher than the polymers as well. As a result, a bioresorbable polymeric scaffold has thicker struts to help compensate for the difference in the material properties to provide a radial stiffness and radial strength this sufficient to provide patency.

Also, the mismatch of the properties of a polymer scaffold and a vessel segment is lower than for a metallic scaffold. This mismatch can be expressed formally in terms of compliance mismatch between the scaffold and the vessel segment at the implant site. The compliance of a material, which is the inverse of stiffness or modulus of a material, refers to the strain of an elastic body expressed as a function of the force producing the strain. The compliance of a scaffold or radial compliance of the scaffold can likewise be defined as the inverse of the radial stiffness of the scaffold. The radial stiffness of the bioresorbable scaffold is lower than a metallic scaffold, so the radial compliance of the bioresorbable scaffold is higher than a metallic scaffold. The compliance mismatch of a polymer scaffold is lower than a metallic stent.

The compliance of a stent, both nondegradable and resorbable, is necessarily much lower than the vessel segment in order for the scaffold to support the vessel at a deployed diameter with minimal periodic recoil due to inward radial forces from the vessel walls. Additionally, it results in better conformity (and less straightening) of the scaffolded segment to the overall curvature of the adjacent segments in the treated vessel. However, an additional aspect of a bioresorbable polymer scaffold that may contribute to favorable clinical outcomes is that the compliance mismatch decreases with time due to the degradation of the bioresorbable polymer. As the polymer of the scaffold degrades, mechanical properties of the polymer such as strength and stiffness decrease and compliance increases. As a result, the radial strength of the scaffold decreases with time and the compliance of the scaffold increases with time since these properties depend on the properties of the scaffold material.

Further embodiments of the present invention include pharmacological approaches to reducing angina that may be used in conjunction with a bioresorbable scaffold or metallic stent. In such embodiments, a scaffold may include a therapeutic agent that reduces angina or PPCP. The scaffold is configured to provide a controlled release of the therapeutic agent upon implantation of the scaffold in a patient.

The therapeutic agents that reduce or prevent angina or PPCP may referred to as anti-angina agents and anti-PPCP agents. The anti-angina or anti-PPCP agents include anesthetic agents, nitrates, beta-blockers, calcium channel blockers, ranexa, nitric oxide donors, nitric oxide, generators, and alpha-adrenergic blockade.

A bioresorbable scaffold or durable stent with an anti-anginal drug may be balloon expandable or self-expanding. The anti-angina or anti-PPCP agents may be included in a coating over a scaffold body or a durable stent. The scaffold body or stent may be balloon expandable or self-expanding. The anti-angina or anti-PPCP agents may be incorporated into the polymer scaffold as an alternative or in addition to a coating over the scaffold.

The scaffold may be used in any artery or vessel in the body in addition to coronary. The scaffold may also have an anti-restenotic drug such as everolimus. Such a scaffold may be implanted in the cerebral, carotid, coronary, aortic, renal, iliac, femoral, popliteal, tibial, or other peripheral vasculature.

The coatings including the anti-angina or anti-PPCP agents may include the agents and a coating polymer. The coating polymer can include any of the polymers or any combination of the polymers disclosed herein. In particular, the polymer is a lactide or lactic acid polymer which comprises poly(lactic acid) or poly(lactide) (“PLA”). In one embodiment, a lactide or lactic acid polymer can be a polymer which incorporates at least 5% (w/w) of L-lactic acid or D-lactic acid. Poly(lactic acid) based polymers (PLA based polymers) include poly(L-lactide), poly(D-lactide), poly(D,L-lactide), poly(D,L-lactide) having a constitutional unit weight-to-weight (wt/wt) ratio of about 96/4, poly(L-lactide-co-D,L-lactide), poly(L-lactide-co-glycolide), poly(D,L-lactide-co-glycolide), poly(L-lactide-co-caprolactone), poly(D,L-lactide-co-caprolactone), and poly(D, L-lactide) made from polymerization of a racemic mixture of L- and D-lactides. In an embodiment, the poly(lactic acid) based polymers (PLA based polymers) include poly(D,L-lactide) having a constitutional unit weight-to-weight (wt/wt) ratio of about 93/7, about 94/6, about 95/5, about 96/4, about 97/3, about 98/2, or about 99/1. The caprolactone copolymers may have 1 to 5 wt % caprolactone units. The coating polymer may also be a blend of any combination of the polymers described herein. The coating polymer may also be a blend of a PLA based polymer and polycaprolactone with about 1 to 5 wt % of polycaprolactone. The term “constitutional unit” refers to the composition of a monomer as it appears in a polymer. The coating polymer can also be a blend of a PLA based polymer and other biocompatible polymers known in the art. The polymer can comprise a copolymer of lactide and glycolide. In an embodiment, the polymer comprises a poly(L-lactide-co-glycolide) copolymer. In an embodiment, the poly(L-lactide-co-glycolide) copolymer is amorphous. In an embodiment, the polymer comprises poly(D,L-lactide), poly(lactide-co-glycolide), polylactide-co-polycaprolactone, poly(L-lactide-co-trimethylene carbonate), polytrimethylene carbonate or copolymers thereof, poly orthoesters or copolymers thereof, poly anhydrides or copolymers thereof, polylactide or copolymers thereof, polyglycolides or copolymers thereof, polycaprolactone or copolymers thereof, or polyiminocarbonates or copolymers thereof. The molecular weight of the coating polymer may be less than 30 kDa, less than 50 kDa, less than 70 kDa, less than 100 kDa, 30 to 40 kDa, 50 to 60 kDa, 60 to 70 kDa, 70 to 80 kDa, 80 to 90 kDa, 90 to 100 kDa, 100 to 120 kDa, 120 to 150 kDa, or greater than 150 kDa.

Thickness or average thickness of the coating including the anti-angina or anti-PPCP agent on the scaffold may be less than 10 microns, less than 5 microns, less than 3 microns, 1 to 10 microns, 1 to 5 microns, 1 to 3 microns, 2.5 microns, 1 to 2 microns, 2 to 3 microns, 2 to 2.5 microns, 2 to 5 microns, 3 to 5 microns, or 5 to 10 microns. The coating may be over part of the surface or the entire surface of a scaffold body. The scaffold may include the drug release coating and the scaffold may be free of drug, aside from any incidental migration of drug into the scaffold from the coating.

The anti-angina or anti-PPCP agent coating layer may additionally include another type of agent such as an antiproliferative (AP) or anti-inflammatory (AI) agent. The drug release rate may be controlled by adjusting the ratio of drug and polymeric coating material. The drug may be released from the coating over a period of one to two weeks, up to one month, or up to three months after implantation. For example, the layer may include an olimus drug which refers to a macrocyclic lactone chemical species which is a derivative, metabolite, or otherwise has a chemical structure similar to that of sirolimus and is useful for the treatment of neointimal hyperplasia, restenosis, and/or other vascular conditions, such as vulnerable plaque. Examples of “olimus drugs” include, but are not limited to, biolimus, everolimus, merilimus, myolimus, novolimus, pimecrolimus, 16-pent-2-ynyloxy-32(S)-dihydro-rapamycin, ridaforolimus, tacrolimus, temsirolimus and zotarolimus.

The dose per unit scaffold length of the anti-angina or anti-PPCP agent on the scaffold may be less than 1 μg/mm, 1 to 7 μg/mm, 1 to 3 μg/mm, 3 to 5 μg/mm, 5 to 7 μg/mm, 7 to 10 μg/mm, 10 to 15 μg/mm, 15 to 25 μg/mm, 1 to 25 μg/mm or greater than 25 μg/mm. The anti-angina/anti-PCP agent may be less than 50 wt %, 10 to 30 wt %, 30 to 50 wt %, 50 to 70 wt %, or greater than 70 wt % of the coating or coating layer.

The ratio of polymer to drug in the coating may be 5:1 to 1:5 or 1:2 to 2:1, where the drug may refer to only the anti-angina/anti-PCP agent or the anti-angina/anti-PCP agent and an AP or AI agent.

The scaffold may further include coating layer that includes only anti-angina or anti-PPCP agent and a layer that includes only an AP or AI agent with the former over the latter or the latter over the former.

Any one or any combination of anti-angina or anti-PPCP agents may be additionally or alternatively incorporated into a scaffold made of a bioabsborable polymer. In such embodiments, the agents may be mixed or dispersed throughout the scaffold within the scaffold polymer. The scaffold may be less than 30 wt %, 1 to 5 wt %, 5 to 10 wt %, or 10 to 30 wt % of the scaffold.

The release profile of any one or any combination of anti-angina or anti-PPCP agents may be adjusted to provide the most beneficial therapeutic effect. The release profile may be such that at least 85% of the drug is released at 2 weeks, 1 month, 2 months, 3 months, 5 months, 7 months, 10 months, or 12 months.

The release profile of any one or any combination of anti-angina or anti-PPCP agents may be a “sustained release” which generally refers to a release profile of a drug that can include zero-order release, exponential decay, step-function release or other release profiles that carry over a period of time, for example, ranging from several days to several years , for example, 5 to 10 days, 10 days to 1 month, 1 to 3 months, 3 to 6 months, 6 to 10 months, or 10 to 12 months. The terms “zero-order release”, “exponential decay” and “step-function release” as well as other sustained release profiles are well known in the art.

The first approach or set of embodiments is directed towards reducing or eliminating PPCP stemming from the causes that may be non-ischemic in origin. For example, the causes may be stretch injury to the coronary, coronary trauma, or dissection and trauma extending out into the adventitia. These causes may be induced by the implantation and/or presence of an implanted scaffold.

Such embodiments include incorporating a local anesthetic (pain killer) on or in a scaffold to provide controlled release with the intent of achieving therapeutic tissue concentrations for a minimum of two weeks to eliminate post procedural chest pain. The tissue concentrations of local anesthetic may be in or adjacent to the vessel at the implant site of the scaffold. The released local anesthetic act on the nerves associated with the chest pain. Local anesthetics refer to agents that cause a reversible loss of sensation for a limited region of the body while maintaining consciousness. Local anesthetics act by binding to fast sodium channels from within (in an open state) and can be either ester or amide based.

The controlled release may be at least 85% of the agent at 2 weeks of implantation. Criteria for the active local anesthetic drug includes approved and preferably generic, potent as the payload in a scaffold coating or in the scaffold itself is limited, and high chemical stability to withstand terminal sterilization via radiation or ETO.

Exemplary useful local anesthetic agents include, but are not limited to, lidocaine, mepivacaine, bupivacaine, levobupivacaine, ropivacaine, etidocaine, prilocaine, and articaine:

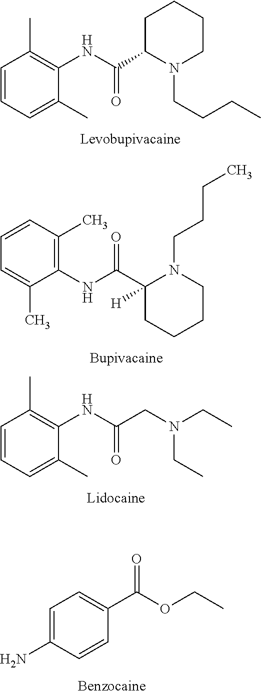 [SMILES: CCCCN1CCCC[C@H]1C(=O)Nc1c(C)cccc1C ; CCCCN1CCCC[C@H]1C(=O)Nc1c(C)cccc1C ; CCN(CC)CC(=O)Nc1c(C)cccc1C ; CCOC(=O)c1ccc(N)cc1]

The amide anesthetics may be are preferred over ester anesthetics as they are more stable. All of these agents block nerve conduction by blocking sodium channels in the nerve cell membrane. Of these, the more potent levobupivacine and bupivacaine may be preferred as they require a smaller dose and are more long lasting. Inspection of their structures show them to be more chemically stable than everolimus.

The local anesthetic agent may be compounded into a drug delivery coating. In such embodiments, the local agent may be included in a polymer coating that includes another drug, such as an anti-inflammatory or antiproliferative'drug. For example, the local anesthetic agent may be included in a poly(D,L-lactide)/everolimus coating.

Bupivacaine is supplied as the hydrochloride salt which may be stable in a coating. Otherwise, it could be formulated with a lipid soluble anion such as caprylate, laurate or stearate. Use of the free base is possible, but oxidation resistance may be reduced.

For a scaffold that includes both anesthetic agents and anti-proliferative (AP) or (AI) agents, the AP or AI agent may be released over a period of two to four months post-implantation and the anesthetic agent may be released over a period of 2 weeks to 1 month. For example, at least 85% of the AP or AI agent may be released at three months post-implantation while the release of the anesthetic agent may be as disclosed herein.

In some embodiments, the coating includes a layer having an anesthetic agent over layer having an AP/AI agent. The anti-angina layer may be devoid of AP/AI agent and the AP/AI agent may be devoid of anti-angina agents. The thickness of the coating layers may be any combination of the thicknesses disclosed herein. The coating polymers for two layers may be the same or different. If one embodiment, the anesthetic layer polymer may be faster degrading than the AP/AI layer polymer.

Inclusion of a local anesthetic into a coronary scaffold or stent coating may reduce or eliminate a large source of post-procedural chest pain, increasing patient comfort and reducing unnecessary health care costs associated with ruling out more serious causes of postprocedural chest pain.

In some embodiments, the dose of the anesthetic agent varies along a length of the stent body such that the dose increases from a proximal to a distal end of the stent. Since native coronary vasculature is tapered, which is most dramatic in a left anterior descending artery (LAD), a higher stretch ratio is imposed on a vessel wall in a distal portion of the scaffold when compared to the proximal scaffold region. Therefore, biasing anesthetic payload distally may more effectively treat pain borne out of circumferential vascular stretch phenomena. The variation may be, for example, in a linear or in a step-wise fashion. The distal 50% of a length of the stent body may have greater than 50%, 51% to 60%, 60 to 70%, 60 to 80%, 60 to 90%, 80 to 95%, 70 to 80%, or greater than 95% of the total drug dose.

Biasing the dose of anesthetic agents distally may further be advantageous given that there is only so much volumetric / surface are on the scaffold, anesthetic biased distally and other anti-angina agents biased proximally could allow for an effective combination that treats stretch pain most efficiently while adding other agents where there is room on the proximal scaffold structure.

Further embodiments include a pharmacological approach to reducing or eliminating chronic angina over a time frame of weeks to 12 months. Such an approach requires a different mechanism of action than an anesthetic since such chronic angina is ischemic in origin. It is preferable for the coronary scaffold to be so effective that there is no restenosis and full restoration of vasoreactivity by one year. However, a finite, cumulative rate of angina is observed at one year, with the rate beyond that appears to taper off. Many agents are used systemically to treat chronic angina and may be used for local delivery on a scaffold. These include nitrates, beta-blockers, calcium channel blockers, ranexa, nitric oxide donors / generators, and alpha-adrenergic blockers.

Embodiments of the invention including one or any combination thereof in or on a scaffold in a manner that provides controlled release of agents to treat chronic angina in a time frame of two weeks to 12 months. The agents are released at or adjacent to the vessel wall at the implant site of the scaffold. In some embodiments, less than 20%, less than 50%, or less than 70% of the anti-angina agent contained in or on the stent is released at 6 months after implantation. In some embodiments, at least 20%, at least 50% or at least 70% are release between 6 months and 1 year after implantation. The controlled release of the agents may treat chronic angina by reducing or eliminating ischemia.

In certain embodiments, the controlled release to treat chronic angina with anti-angina agents is performed with no systemic treatment with anti-angina agents. In other embodiments, the controlled release to treat chronic angina with anti-angina agents is performed in addition to systemic treatment with anti-angina agents.

For a scaffold that includes both anti-angina agents and AP or AP agents, the AP or AI agent may be released over a period of two to four months post-implantation and the anti-angina agent may be released over a period of 2 weeks to 12 months. For example, at least 85% of the AP or AI agent may be released at three months post-implantation while the release of the anti-angina agent may be as disclosed herein.

Exemplary coating polymers for use with anti-angina agents for treating chronic angina include poly(L-lactide), poly(D-lactide), poly(D,L-lactide), poly(D,L-lactide) having a constitutional unit weight-to-weight (wt/wt) ratio of about 96/4, poly(L-lactide-co-D,L-lactide), poly(L-lactide-co-glycolide), poly(D,L-lactide-co-glycolide), poly(L-lactide-co-caprolactone), poly(D,L-lactide-co-caprolactone), poly(D, L-lactide) made from polymerization of a racemic mixture of L- and D-lactides. poly(L-lactide) (PLLA), poly(D-lactide) (PDLA), polymandelide (PM), polyglycolide (PGA), poly(L-lactide-co-D,L-lactide) (PLDLA), poly(D,L-lactide) (PDLLA), poly(caprolactone-co-L-lactide), poly(caprolactone-co-D,L-lactide), poly(caprolactone-co-glycolide), poly(D,L-lactide-co-glycolide) (PLGA) and poly(L-lactide-co-glycolide) (PLLGA), polycaprolactone (PCL), poly(trimethylene carbonate) (PTMC), polydioxanone (PDO), poly(4-hydroxy butyrate) (PHB), and poly(butylene succinate) (PBS), poly(L-lactide)-b-polycaprolactone (PLLA-b-PCL) or poly(L-lactide)-co-polycaprolactone (PLLA-co-PCL), poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, polyethylene amide, polyethylene acrylate, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, ethylene vinyl alcohol copolymer (commonly known by the generic name EVOH or by the trade name EVAL®), poly(butyl methacrylate), poly(vinylidene fluoride-co-hexafluoropropylene), polyvinylidene fluoride, ethylene-vinyl acetate copolymers, and polyethylene glycol.

In some embodiments, the coating includes a layer having the agent to treat chronic angina and a layer having an AP/AI agent. The AP/AI layer may be over the angina layer. The anti-angina layer may be devoid of AP/AI agent and the AP/AI agent may be devoid of anti-angina agents. The thickness of the coating layers may be any combination of the thicknesses disclosed herein. The coating polymers for two layers may be the same or different. If one embodiment, the anti-angina layer polymer may be slower degrading than the AP/AI layer polymer.

Beta blockers are currently prescribed for stable angina. Beta blockers have systemic effects on the adrenergenic receptors which reduce heart rate and lowers myocardial oxygen consumption. It is believed that local delivery from a scaffold may have none of these systemic effects.

A calcium channel blocker (CCB) disrupts the movement of calcium ions (Ca²⁺) through calcium channels from outside to the inside of cells. By acting on arterial smooth muscle cells, the reduce contraction of the arteries and cause an increase in arterial diameter. As a result, they act as vasodilators. Their action on cardiac myocytes is to reduce the force of contraction of the heart. An effect on cardiac nerves is to reduce the electrical activity of the heart and slow the heartbeat.

Despite their effectiveness, systemic use of CCB's often have a high mortality rate over extended periods of use, and have been known to have multiple side effects. However, since these troublesome characteristics are related to systemic use, it is believed that the use CCB's in local delivery may result in a reduction or none of these. characteristics.

Exemplary CCB's include dihydropyridines, lacidipine, amlodipine, nicardipine, nefedipine, and felodipine.

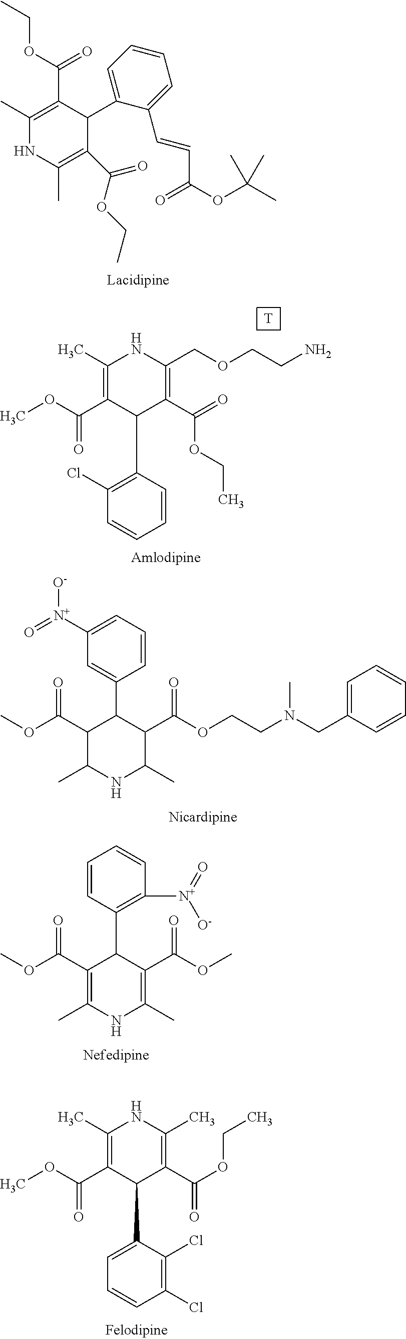

The dihydropyridines are potent vasodilators which makes them advantageous for local delivery to treat very local ischemia that is present, or distal, to a scaffolded coronary segment. “Very local” may refer to a location within 5 mm, 1 to 5 mm , 5 to 10 mm, 10 to 15 mm, 5 to 20 mm, 5 to 30 mm, 10 to 20 mm, 10 to 30 mm, 15 to 20 mm, or 20 to 30 microns from a distal end of the scaffold.

Lacipidine is hydrophobic and restores endothelium dependent vasodilation which makes it one candidate. Amlodipine is another candidate due to its potent vasodilatory effects.

All of these agents are indicated as anti-anginal drugs and they are generic. Nefedipine requires one of the lower oral dosages so may be more preferable or appropriate for local delivery with a scaffold than other drugs that require higher dosages. These drugs are all organic soluble and could be incorporated into a scaffold coating polymer.

Of the many classes of calcium channel blockers, the phenylalkylamine CCB's are more selective for the myocardium, reduce myocardial oxygen demand and reverse coronary vasospasm, and are often used to treat angina. These include:

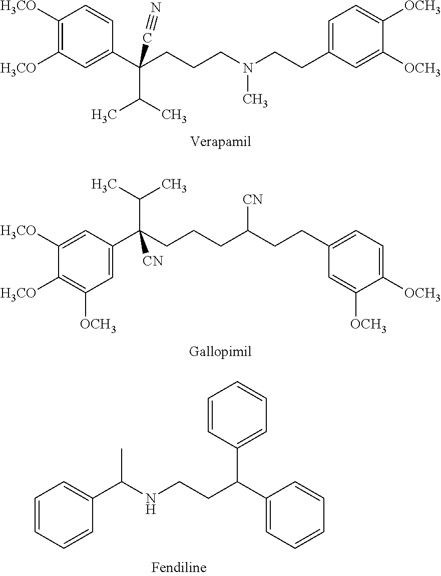 [SMILES: COc1ccc(CCN(C)CCC[C@@](C#N)(c2ccc(OC)c(OC)c2)C(C)C)cc1OC ; COc1ccc(CCC(C#N)CCC[C@](C#N)(c2cc(OC)c(OC)c(OC)c2)C(C)C)cc1OC ; CC(NCCC(c1ccccc1)c1ccccc1)c1ccccc1]

They have minimal vasodilatory effects compared with dihydropyridines cited above, and therefore cause less reflex tachycardia, which should not be a problem for low overall dose, and local drug delivery. The phenylalkylamine CCB's have attractive properties for local delivery, such as liphophilicity and organic solubility. If the amine salt were made with a hydrophobic anion, their stability should be good. Due to their potency, oral dosages of 100-200 mg per day are used since oral bioavailability may require this dose. Lower doses may be used for local delivery.

Nitric oxide donors and generators may also be used in local delivery from a scaffold to treat angina. Nitric oxide donors are molecules that release nitric oxide upon breakdown or dissociation and are disclosed in Guo, Am J Physiol 269: H1122-31, 1995). Nitric oxide generators are catalysts that are capable of catalyzing the generation of nitric oxide and are disclosed in US 20120034222. The resultant nitric oxide will serve as a vasodilator, an inhibitor of smooth muscle cell migration/proliferation, and an inhibitor of platelet adhesion/aggregation. The donor or generator could be applied to the surface of the scaffold, incorporated into coating on the scaffold for short term delivery and treatment or incorporated into the polymeric scaffold for longer term delivery and treatment.

Another embodiment of treating angina or PPCP focuses on addressing reduced flow reserve due to the sympathetic activation and subsequent restriction of arterioles caused by stent implantation. The treatment may be relevant in the short term time frame or over weeks to months following stenting, for example, 1 to 2 weeks, 2 weeks to 1 month, 1 to 2 months or 2 to three months. Gregorini et al (Circulation. 2002; 106:2901-2907) demonstrated that stent implantation caused impairment of flow reserve due to alpha-adrenergic activation immediately after stenting. This may be due to stretch of the artery eliciting a sympathetic constrictor tone or ischemia inducing a reflex increase in sympathetic tone.

Agents that block alpha adrenergic-mediated arteriole vasoconstriction may be delivered from a scaffold to block this sympathetic tone and increase flow reserve, thereby having an anti-anginal or PPCP effect. These agents may be released via a drug delivery coating on a scaffold. Non-selective blockade may be preferred due to biological efficacy. However, selective alpha-1, alpha-2 blockade, or both may be effective and may be preferred for chemical stability or formulation from a device. Non-selective alpha adrenergic blockade agents include: phenoxybenzamine, phentolamine, trazodone, tolazine. Selective blockade agents for alpha-1 blockage include prazosin and doxazosin. Selective blockade agents for alpha-2 blockage include idazoxan and yohimbine.

The prevailing mechanism of degradation of many bioabsorbable polymers is chemical hydrolysis of the hydrolytically unstable backbone. In a bulk degrading polymer, the polymer is chemically degraded throughout the entire polymer volume. As the polymer degrades, the molecular weight decreases. The reduction in molecular weight results in changes in mechanical properties (e.g., strength) and stent properties. For example, the strength of the scaffold material and the radial strength of the scaffold are maintained for a period of time followed by a gradual or abrupt decrease. The decrease in radial strength is followed by a loss of mechanical integrity and then erosion or mass loss. Mechanical integrity loss is demonstrated by cracking and,by fragmentation. Enzymatic attack and metabolization of the fragments occurs, resulting in a rapid loss of polymer mass.

The behavior of a bioabsorbable scaffold upon implantation can divided into three stages of behavior. In stage I, the stent provides mechanical support. The radial strength is maintained during this phase. Also during this time, chemical degradation occurs which decreases the molecular weight. In stage II, the scaffold experiences a loss in strength and mechanical integrity. In stage III, significant mass loss occurs after hydrolytic chain scission yields water-soluble low molecular weight species.

The scaffold in the first stage provides the clinical need of providing mechanical support to maintain patency or keep a vessel open at or near the deployment diameter. In some treatments, the patency provided by the scaffold allows the stented segment of the vessel to undergo positive remodeling at the increased deployed diameter. Remodeling refers generally to structural changes in the vessel wall that enhances its load-bearing ability so that the vessel wall in the stented section can maintain an increased diameter in the absence of the stent support. A period of patency is required in order to obtain permanent positive remodeling.

The manufacturing process of a bioabsorbable scaffold includes selection of a bioabsorbable polymer raw material or resin. Detailed discussion of the manufacturing process of a bioabsorbable stent can be found elsewhere, e.g., U.S. Patent Publication No. 20070283552. The fabrication methods of a bioabsorbable stent can include the following steps:

(1) forming a polymeric tube from a biodegradable polymer resin using a method such as extrusion, injection molding, spraying a polymer solution over a mandrel, or dipping a mandrel into a polymer solution

(2) processing the tube to increase radial strength which can include annealing above a Tg of the polymer, solvent-induced crystallization, radially deforming the tube above the Tg, or any combination thereof,

(3) forming a stent scaffolding from the processed tube by laser machining a stent pattern in the deformed tube with laser cutting, in exemplary embodiments, the strut thickness can be 80-200 microns, or more narrowly, 90-180, 100-160, or 110-140 microns,

(4) optionally forming a therapeutic coating over the scaffolding,

(5) crimping the stent over a delivery balloon, and

(6) sterilization with electron-beam (E-beam) radiation.

Poly(L-lactide) (PLLA) is attractive as a stent material due to its relatively high strength and rigidity at human body temperature, about 37° C. Since it has a glass transition temperature between about 60 and 65° C. (Medical Plastics and Biomaterials Magazine, March 1998), it remains stiff and rigid at human body temperature. This property facilitates the ability of a PLLA stent scaffold to maintain a lumen at or near a deployed diameter without significant recoil (e.g., less than 10%). In general, the Tg of a semicrystalline polymer can depend on its morphology, and thus how it has been processed. Therefore, Tg refers to the Tg at its relevant state, e.g., Tg of a PLLA resin, extruded tube, expanded tube, and scaffold.

In general, a scaffold can be made of a bioresorbable aliphatic polyester. Additional exemplary biodegradable polymers for use with a bioabsorbable polymer scaffolding include poly(D-lactide) (PDLA), polymandelide (PM), polyglycolide (PGA), poly(L-lactide-co-D,L-lactide) (PLDLA), poly(D,L-lactide) (PDLLA), 96/4 poly(D,L-lactide) (PDLLA), poly(D,L-lactide-co-glycolide) (PLGA), poly(L-lactide-co-caprolactone), and poly(L-lactide-co-glycolide) (PLLGA). The poly(L-lactide-co-caprolactone) may have 1 to 5% (by mole or weight) of caprolactone.

With respect to PLLGA, the stent scaffolding can be made from PLLGA with a mole% of GA between 5-15 mol%. The PLLGA can have a mole% of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used. Many other examples can be provided, such as those found in Polymeric Biomaterials, second edition, edited by Severian Dumitriu; chapter 4.

Polymers that are more flexible or that have a lower modulus than those mentioned above may also be used. Exemplary lower modulus bioabsorbable polymers include, polycaprolactone (PCL), poly(trimethylene carbonate) (PTMC), polydioxanone (PDO), poly(4-hydroxy butyrate) (PHB), and poly(butylene succinate) (PBS), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of the blend can include 1-5 wt % of low modulus polymer.

A scaffold may also be made from a tyrosine-derived polycarbonate. These degradable polymers are derived from the polymerization of desaminotyrosyl-tyrosine alkyl esters. J. of Appl. Polymer Sci., Vol. 63, 11, pp. 1467-1479. In the synthesis of tyrosine-derived polycarbonates, L-tyrosine and its natural metabolite desaminotyrosine [3-(4-hydroxphenyl) propionic acid] are used as building blocks to form desaminotyrosyl-tyrosine alkyl esters. A representative structure of a tyrosine-derived polycarbonate is:

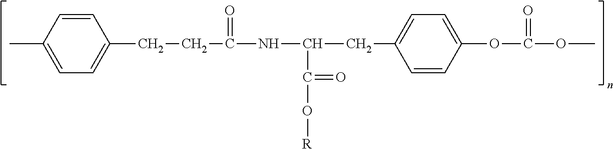

When R is a hydrogen, the repeat unit is desamino-tyrosyl-tyrosine, referred to as “DT.” The pendent group (R) of the polycarbonates can also be, for example, ethyl, butyl, hexyl, octyl, and benzyl esters. The corresponding polymers are referred to as poly(DTE carbonate), poly(DTB carbonate), poly(DTH carbonate), poly(DTO carbonate), and poly(DTBzI carbonate), respectively. The ethyl pendent group may be preferred at least for the reason that the pendent groups are not biodegradable and a shorter pendent group is more easily and safely eliminated by the body.

The bioresorbable scaffold may also be made from poly-anhydride ester. The polyanhydrides ester may be based on salicylic acid and adipic acid anhydride.

The bioresorbable scaffold may be made from bioerodible metals or metal alloys including magnesium, iron, zinc, tungsten, and alloys including these metals.

A durable or non-degradable stent may be made metals including platinum, stainless steel, and nickel-titanium alloys.

The BVS scaffolds are coated with a polymer mixture that includes everolimus, an antiproliferative agent. In general, the anti-proliferative agent can be a natural proteineous agent such as a cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I1, actinomycin X1, and actinomycin C1), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Illinois), prodrugs thereof, co-drugs thereof, and combinations thereof.

An anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, and genetically engineered epithelial cells. The foregoing substances are listed by way of example and are not meant to be limiting. Other active agents which are currently available or that may be developed in the future are equally applicable. The scaffold can exclude any of the drugs disclosed herein.

“Baseline” refers to a time immediately after deployment of a scaffold to a target diameter in a vessel or at a time after deployment long enough to make measurements on the newly deployed scaffold.

“Molecular weight” can refer to number average molecular weight (Mn) or weight average molecular weight (Mw). Molecular weight values may refer to that obtained from Gas Permeation Chromotography using polystyrene reference standards.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through molecular movement. Tg of a given polymer can be dependent on the heating rate and can be influenced by the thermal history of the polymer as well as its degree of crystallinity. Furthermore, the chemical structure of the polymer heavily influences the glass transition by affecting mobility.

The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20 ° C./min heating rate.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to a change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. The modulus typically is the initial slope of a stress—strain curve at low strain in the linear region.

The present invention includes any combination of the embodiments or claims disclosed herein.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method of treating coronary artery disease (CAD) in a patient in need thereof comprising:
selecting a patient in need of treatment of CAD having a lesion in a blood vessel that is an indicator of high risk or a susceptibility of the patient to angina or non-ischemic thoracic chest pain; and
implanting a bioresorbable stent at the lesion in a blood vessel of the patient, wherein the implanted scaffold treats the CAD.

2. The method of claim 1, wherein the lesion is a long diffuse lesion having a length of at least 20 mm.

3. The method of claim 1, wherein the stent is a bioresorbable polymer stent.

4. The method of claim 1, wherein the lesion is an ostial lesion.

5. The method of claim 4, wherein the ostial lesion begins within Ito 5 mm of an origin of a major epicardial artery.

6. The method of claim 1, wherein the lesion is a vulnerable plaque suspect lesion.

7. The method of claim 6, wherein the lesion has less than 50% occlusion as shown by angiography.

8. The method of claim 1, wherein the lesion is a bifurcated lesion

9. The method of claim 1, wherein the patient experiences no angina or non-ischemic thoracic chest pain for at least 1 year after implantation.

10. The method of claim 1, wherein the susceptibility comprises a history of angina of the patient within one year prior to implantation.

11. The method of claim 1, wherein the susceptibility comprises a % diameter stenosis of greater than 70% at a site of implantation of the stent.

12. A method of treating coronary artery disease (CAD) in a patient or population of patients comprising:
recommending treatment or describing advantages relating to reduced angina of bioresorbable polymer stents or a type of bioresorbable stents for treating CAD for a patient or patient population with factors, conditions, or characteristics, or any combination thereof which makes the patient or population of patients susceptible to angina, wherein the recommending or describing includes communicating electronic ally or in printed; and
providing a plurality of the bioresorbable stents or type of stents to a medical facility, medical professional, or distributer for distribution to a medical facility or medical professional for treatment of a patient or population of patients in need of treatment of the CAD that has or does not have one or more of the factors, conditions, or characteristics.

13. The method of claim 12, wherein a statistically significant number of the population of patients experiences lower frequency, severity, or diagnosis rate of angina than has been shown for a metal platform stent.

14. The method of claim 12, wherein the recommended bioresorbable stent has been shown to provide a reduced rate of angina as compared to a durable metal platform stent in other patient populations.

15. The method of claim 12, wherein the recommendation is made as an alternative to a metal platform stent.

16. The method of claim 12, wherein the advantages comprise reduced angina from treatment with the bioresorbable polymer stent as compared to a metal platform stent.

17. The method of claim 12, further comprising providing or sending the bioresorbable stent to a medical facility, medical professional, or distributer for distribution to a medical facility or medical professional for treatment of a patient or population of patients in need of treatment of the CAD that has or does not have one or more of the factors, conditions, or characteristics.

18. The method of claim 12, further comprising implanting the bioresorbable polymer stents in a patient or population of patients in need of treatment of the CAD that has or does not have one or more of the factors, conditions, or characteristics, wherein the implanted stent treats the CAD and the patient or population of patients experiences no angina or non-ischemic chest pain or a reduced degree of angina or non-ischemic chest pain as compared to a metallic stent during at least the first 30 days after implantation.

19. The method of claim 12, wherein the factors, conditions, or characteristics are selected from the group consisting type of coronary lesion, suffering from a CAD-related condition or non-CAD disease, race, ethnicity, gender, and any combination thereof.

20. The method of claim 19, wherein the type of coronary lesions is selected from the group consisting of bifurcated lesion, long diffuse lesion, ostial lesion, and vulnerable plaque suspect lesion (<50% occlusion by angiography).

21. The method of claim 19, wherein the CAD-related condition or non-CAD disease is selected from the group consisting of suffering diabetes, obesity, prone to vasospasm, and any combination thereof.

22. The method of claim 19, wherein the race or ethnicity comprises Indian sub-continent descent.

23. A method of treating coronary artery disease (CAD) in a patient or population of patients comprising:
identifying a patient or population of patients in need of treatment of CAD; and
implanting the bioresorbable polymer stent in the patient or population of patients for treating the CAD,
wherein the stent is implanted in a stenotic segment of a blood vessel in the patient or population of patients,
wherein during a first period of at least 30 days after implanting when the mechanical properties of the stent are minimally unaffected by degradation the stent exhibits reduced stress-strain interactions with the vessel as compared to a metal platform stent due to greater axial conformability, circumferential conformity, reduced medial compression, higher stent area:artery ratio, or any combination thereof,
wherein during a second period after the first period, stress-strain interactions with the vessel are reduced due to degradation of the stent resulting in a decrease in radial strength of the stent and loss of mechanical integrity of the stent both of which increase the vessel freedom of movement,
wherein the increase in freedom of movement of the vessel allows for pulsatility in the vessel and optionally positive remodeling of the vessel during the second period, and
wherein angina is reduced in the patient or the population of patients as compared to a metal platform stent or prevented during the first and/or second period due to one or any combination of the reduced stress-strain interactions in the first period, the increased pulsatility during the second period, and the positive remodeling during the second period.

24. The method of claim 23, wherein the increased pulsatility and the positive remodeling additively enhance blood flow rate which reduces angina in the patient or the population of patients as compared to a metal platform stent, wherein the increased pulsatility and the positive remodeling increase the blood flow rate as compared with a reference flow rate for a stented vessel and wherein the increased pulsatility and the positive remodeling additively enhance blood flow rate between 6 and 12 months post-implantation.

25. The method of claim 23, wherein the increased pulsatility enhances blood flow rate which reduces angina in the patient or the population of patients as compared to the metal platform stent prior to or in the absence of the positive remodeling, and wherein the increased pulsatility and reduction in angina starts at about 6 months.

26. The method of claim 23, wherein the reduced strain-strain interactions in the first period provides optimal stress-strain equilibration during the first period which reduces angina in the patient or the population of patients as compared to a metal platform stent while maintaining patency.

27. The method of claim 23, wherein the reduced strain-strain interactions in the first period and the increased pulsatility promote, additively or synergistically, functional neo media/endothelium, resulting in benign positive remodeling, and wherein the reduced strain-strain interactions in the first period and the increased pulsatility promote, additively or synergistically, functional neo media/endothelium starts at about 6 months post implantation.

28. A medical device, comprising:
a bioabsorbable stent body; and
a coating layer comprising a bioabsorbable coating polymer and an anesthetic agent, the bioresorbable coating polymer having a number average molecular weight less than 200 kDa, the coating layer having a thickness of 1 to 10 microns, wherein a dose per unit stent body length of the anesthetic agent on the stent body is 1 to 25 g/mm.

29. The medical device of claim 28, wherein upon implantation of the medical device in a patient, the coating and dose of the anesthetic agent provides a release of the anesthetic agent effective to reduce or eliminate post-procedural chest pain in the patient during at least the first two weeks post-implantation.

30. The medical device of claim 28, wherein the anesthetic agent is selected from the group consisting of lidocaine, mepivacaine, bupivacaine, levobupivacaine, ropivacaine, etidocaine, prilocaine, articaine, and any combination thereof.

31. The medical device of claim 28, wherein the bioabsorbable stent body comprises a material selected from the group consisting of a lactide-based polymer, a bioerodible metal, and a tyrosine-based polycarbonate polymer.

32. The medical device of claim 28, wherein at least 85% of the anesthetic is released at 1 month after implantation.

33. The medical device of claim 28, wherein the coating layer further comprises an antiproliferative agent.

34. A medical device, comprising:
a bioabsorbable stent body; and
a coating layer, the coating layer comprising of a bioabsorbable coating polymer and an anti-angina agent effective to reduce or eliminate ischemia induced chest pain in a patient selected from the group consisting of calcium channel blockers, nitric oxide donors, nitric oxide generators, and alpha-adrenergic blockade agents, wherein a number average molecular weight of the bioabsorbable coating polymer is less than 200 kDa, the coating layer having a thickness of 1 to 10 microns.

35. The medical device of claim 34, wherein upon implantation of the medical device in the patient, the coating and dose of the anti-angina agent provides a release of the anti-angina agent effective to reduce or eliminate ischemia induced post-procedural chest pain in the patient between 6 months and 1 year.

36. The medical device of claim 34, wherein a dose per unit stent body length of the anti-angina agent on the stent body is 1 to 25 μg/mm.

37. The medical device of claim 34, wherein less than 50% of the anti-angina agent is released at 6 months after implantation.

38. The medical device of claim 34, wherein the calcium channel blockers are selected from the group consisting of dihydropyridines, lacidipine, amlodipine, nicardipine, nefedipine, felodipine, and phenylalkylamines.

39. The medical device of claim 38, wherein the phenylalkylamines are selected from the group consisting of verapamil, gallopimil, and fendiline.

40. The medical device of claim 38, wherein the alpha adrenergic blockers are selected from the group consisting of non-selective alpha blockade agents and selective alpha blockade agents.

41. The medical device of claim 40, wherein non-selective alpha adrenergic blockade agents are selected from the group consisting of phenoxybenzamine, phentolamine, trazodone, and tolazine.

42. The medical device of claim 40, wherein the selective blockade agents are selective for alpha-1 blockage and are selected from the group consisting of prazosin and doxazosin.

43. The medical device of claim 40, wherein the selective blockade agents are selective for alpha-2 blockage and are selected from the group consisting of idazoxan and yohimbine.

44. The medical device of claim 34, wherein the coating layer further comprises an antiproliferative agent.
